(19) United States
(12) Patent Application Publication
(10) Pub. No.: US 2005/0112638 A1
(43) Pub. Date: May 26, 2005

(54) Methods and agents for regulating angiotensin activity

(75) Inventor: Kathryn Sandberg, Silver Spring, MD (US)

Correspondence Address: FISH & NEAVE IP GROUP;ROPES & GRAY LLP, ONE INTERNATIONAL PLACE, BOSTON, MA, 02110-2624, US

(73) Assignee: Georgetown University, Washington, DC (US)

(21) Appl. No.: 10/951,091

(22) Filed: Sep. 27, 2004

Related U.S. Application Data
(60) Provisional application No. 60/506,191, filed on Sep. 26, 2003.

(51) Int. Cl. C12Q 1/68; A61K 48/00; A61K 31/401

(52) U.S. Cl. 435/6; 514/44; 514/423

(57) ABSTRACT

In certain aspects, the present invention relates to methods and preparations for treating angiotensin II-mediated diseases, and in particular, methods of screening for agents that modulate post-transcriptional regulation of angiotensin II receptors and the use of such agents.

### RELATED APPLICATION

This application claims the benefit of the filing date of U.S. Provisional application No. 60/506,191, entitled Methods and Agents for Regulating Angiotensin Activity and filed Sep. 26, 2003. The entire teachings and specification of the referenced application are incorporated herein by reference.

### FUNDING

Work described herein was funded, in whole or in part, by the National Institutes of Health RO1 Grant #HL57502. The United States government has certain rights in the invention.

### BACKGROUND OF THE INVENTION

The renin-angiotensin system (“RAS”) plays an integral role in maintaining vascular tone, optimal salt and water homeostasis, and cardiac function in humans. Angiotensin II is a peptide hormone produced mainly in the blood during the cleavage of angiotensin I by angiotensin-converting enzyme (“ACE”) which is localized on the endothelium of blood vessels of lung, kidney, and many other organs. Angiotensin II interacts with specific receptors on the surface of the target cells. Two receptor subtypes have been identified: ATI receptors (“ATIRs”) and AT2 receptors (“AT2Rs”). The type-I angiotensin II receptor (“ATIR”) is a G protein-coupled receptor and mediates most of the biological actions of angiotensin II through activation of a phosphatidylinositol-calcium second messenger system (Murphy et al., Nature 1991; 351: 233-236). In recent years, it has been recognized that pathologic consequences may result from overactivity of angiotensin II-mediated signaling pathways. Examples of angiotensin II-mediated diseases include renal artery stenosis, hypertension, diabetic and nondiabetic nephropathies, left ventricular hypertrophy, coronary atherosclerosis, myocardial infarction, and congestive heart failure (Brewster et al., Am. J. Med. Sci. 2003; 326: 15-24).

Recently, considerable efforts have been made to identify substances that bind selectively to the AT1R. These active compounds are called angiotensin II antagonists or angiotensin II receptor blockers (“ARBs”). As a consequence of the inhibition of the AT1R, these antagonists can, for example, be employed as anti-hypertensives or for treating congestive heart failure (Ramahi, Postgrad. Med 2001; 109:115-122). The recently developed class of ARBs appear to be as effective as ACE inhibitors in delaying the progression of renal injury in animal models of diabetes (Barnett, Blood Press 2001; 10 Suppl 1:21-26). They act by selectively blocking the binding of angiotensin II to ATIR and may therefore offer a more complete blockade of RAS than ACE inhibitors, which inhibit the conversion of angiotensin I to angiotensin II. With the ATIR blocked, angiotensin II is available to activate the type-2 angiotensin receptor (“AT2R”), which mediates several potentially beneficial effects in the cardiovascular system, including vasodilation, antiproliferation, and apoptosis (Siragy, Am J Cardiol. 1999; 84:3S-8S).

Several polymorphisms in the human AGTR1 gene have been discovered, some of which have been reported to be associated with hypertension. For example, Bonnardeaux et al. (Hypertension 1994; 24:63-69) identified an adenine or cytosine polymorphism (A1166C) located in the 3-prime untranslated region of the AT1R gene. This variant was present at a significantly elevated frequency in 206 Caucasian patients with essential hypertension. Wang et al. (Clin. Genet. 1997; 51:31-34) did a case-control study of the A1166C variant in a well-characterized group of 108 Caucasian hypertensive subjects with a strong family history (two affected parents) and early onset disease. The frequency of the A1166C allele in this subject group was 0.40 in hypertensives compared to 0.29 in normotensives. Further characterization of the A1629C polymorphism has shown it is significantly more frequent in women who develop pregnancy-induced hypertension as compared to healthy controls (Nalogowska-Glosnicka et al., Med Sci. Monit. 2000; 6:523-529). These data further support the notion that AT1R is an important target for the control of angiotensin II-dependent hypertension.

Angiotensin II has also been implicated in the development of cardiac hypertrophy, because ACE inhibitors and ARBs prevent or regress ventricular hypertrophy in animal models and in humans. Herzig et al. studied AT I R promoter activity during cardiac hypertrophy, and discovered that AT1R expression is enhanced 160% in hypertrophied myocardium compared to normal myocardium (Proc. Natl. Acad. Sci. U.S.A 1997; 94:7543-7548).

The ATIR gene is located on chromosome 3q21-q25 and contains 1 exon that encodes a 359 amino acid protein. Two human AT1R subtypes have been identified, and recent evidence has indicated there may be as many four ATIR splice variants that are expressed in humans (Martin et al., Mol. Endocrinol. 2001; 15:281-293). Reference sequences for the AT1R gene (Genaissance Reference No. 2506603; SEQ ID NO: 1), coding sequence (GenBank Accession No:NM000685.2).

In all angiotensin receptors thus far cloned, the coding region of the receptor is contained within one exon. Genomic analysis of mouse, rat, and human angiotensin receptors, suggests that alternative splicing within the 5′ leader sequence (“5′LS”) is a common event in mammalian AT1Rs and AT2Rs. The rat AT1R gene has 3 exons, and two distinct alternatively spliced transcripts are expressed in rat tissues. Human ATIR has also been shown to have 4 exons, and 4 distinct alternatively spliced transcripts expressed in human tissues in varying abundance. Curnow (Clin. Exp. Pharmacol. Physiol. Suppl. 1996; 3:S67-73) and Curnow et al. (Mol. Endocrinol. 1995, 9:1250-62) showed that the human exon 2-containing transcript had a markedly lower translation of the downstream open reading frame compared to the transcripts lacking exon 2 and suggested that the inhibitory effect of exon 2 on translation was due to a minicistron commencing with an ATG in an optimal context for translation initiation. Warnecke et al. (J. Mol. Med. 1999, 77: 718-27) analyzed the alternatively spliced AT1R transcripts in endomyocardial biopsies and found that in failing hearts, the percentage of exon-containing transcripts (out of the total AT1R mRNA) was significantly reduced in atria and in the left ventricle. There is a need to develop agents and methods that can regulate the translation and expression of AT1R splicing variants.

### SUMMARY OF THE INVENTION

This invention relates to methods and preparations for treating angiotensin II-mediated diseases, and in particular, methods of screening for agents that modulate post-transcriptional regulation of angiotensin II receptors and the use of such agents. As described herein, it has been determined that alternative splicing of the AT1R gene plays an important role in regulating its translation and expression of AT1R proteins. The alternatively spliced transcript lacking exon 2 shows enhanced translation and results in higher density of AT I R proteins expressed in the respective cell membrane, relative to the alternatively spliced transcript including exon2. Also described herein is the finding that the 5 LS of exon 2 appears to contain a cis element conferring translational regulation and that RNA-binding proteins (RNABPs) may specifically interact with the 5′LS of AT1R mRNA and thereby regulate AT1R translation.

The present invention is directed to methods and preparations relating to angiotensin II-mediated diseases. Specifically, the invention relates to methods of screening for agents that regulate angiotensin receptor genes at the post-transcriptional level, pharmaceutical preparations comprising such agents, and uses of such agents in treating angiotensin II-mediated diseases.

In one aspect, the present invention provides methods of screening for agents that regulate alternative splicing of AT 1 R gene. In a specific embodiment, the screening method identifies agents that increase the ratio of exon 2-containing transcript (“E2” or “E1,2,3”) to the transcript without exon 2 (“AE2” or “E1,3”), and in a further embodiment, the agents increase the ratio of E2/AE2 without changing the total amount of AT1R mRNA. In another embodiment, the screening method identifies agents that promote alternative splicing resulting in E2 or inhibit alternative splicing resulting in AE2 or both, and in a specific embodiment, the agents increase the ratio of E2/AE2 without changing the total amount of AT1R mRNA.

In a specific embodiment, siRNAs are candidate agents to be screened. In particular embodiments, siRNAs targeting the junction between exons 1 and 3, which is unique to AE2, are the candidate agents to be screened.

In a specific embodiment, small molecules such as small molecules are generated by combinatorial synthesis, are candidate agents to be screened.

Yet another aspect of the screening method identifies agents that selectively inhibit translation of the ΔE2 transcript. In particular embodiments, the agents are antisense polynucleotides that specifically attenuate the expression of ΔE2 transcript.

Another aspect of the present invention is directed to an expression vector comprising a nucleic acid encoding an antisense molecule, an siRNA molecule, or a peptide that regulates AT1R post-transcriptionally.

Another aspect of the invention is directed to transgenic animals. The subject transgenic animal may have attenuated expression of native AT1R gene due to a transgene encoding an antisense molecule, or an siRNA molecule of the present invention. Alternatively, the transgenic animal may express AT1R from a transgene encoding the E2 or ΔE2 transcript. In a particular embodiment, the native AT1R gene is replaced through homologous recombination by a transgene encoding the E2 or ΔE2 transcript.

Another aspect of the invention relates to the discovery that specific RNA-binding proteins (RNABPs) regulate the translation of AT1R mRNA. In one embodiment, the subject screening method identifies agents that modulate the activities of the RNABPs, and thereby regulate the translation of AT1R transcripts. In a further embodiment, the RNABPs that inhibit translation of ΔE2 serve as targets to screen for agents that facilitate the inhibitory effect of these RNABPs on translation. The agents that target the RNABPs can be small molecules, proteins, antibodies, nucleic acids encoding polypeptides that modulate the RNABPs, or generally agents that promote the function of these RNABPs.

A further aspect of the invention is directed to gene therapy utilizing a desired agent of the present invention. Examples without limitation include an RNAi construct of the present invention.

In certain embodiments, the candidate agents regulating AT1R mRNA translation and expression do not affect translation and/or expression of the AT2R gene. In other embodiments, the candidate agents regulating AT1R mRNA translation and expression may promote expression of the AT2R gene post-transcriptionally, i.e., either enhancing translation or facilitating expression or both.

Another aspect of the present invention provides pharmaceutical preparations comprising the agents having the recited property, for example, capable of increasing the ratio of E2/ΔE2 without changing the total amount of AT1R mRNA.

In a particular embodiment, the present invention provides a vector suitable for gene therapy comprising siRNAs having recited properties.

In one embodiment, the present invention provides a vector comprising nucleic acids encoding polypeptides that modulate the RNABP's activities.

Another aspect of the present invention relates to the use of the agents as discussed above to treat angiotensin II-mediated diseases. In one embodiment, the subject method comprises administering to a patient a pharmaceutical preparation comprising such agents. In certain embodiments, the patient suffers from a condition caused by hypertension or cardiac hypertrophy.

Still another aspect of the present invention provides a method of conducting a pharmaceutical business comprising:
a). identifying an agent that increases the ratio of E2/ΔE2, promotes the translation of E2, or inhibits the translation of ΔE2;
b). conducting therapeutic profiling of the agent identified in step (a) for efficacy and toxicity in animals; and
c). formulating a pharmaceutical preparation including one or more agents identified in step (b) as having an acceptable therapeutic profile.

Preferably, the method of conducting a pharmaceutical business further includes establishing a distribution system for distributing the pharmaceutical preparation for sale, and (optionally) establishing a sales group for marketing the pharmaceutical preparation.

Yet still another aspect of the present invention provides a method of conducting a pharmaceutical business comprising:
a). identifying an agent that increases the ratio of E2/ΔE2, promotes the translation of E2, or inhibits the translation of ΔE2;
b). (optionally) conducting therapeutic profiling of the agent identified in step (a) for efficacy and toxicity in animals; and
c) licensing, to a third party, the rights for further development of the agent.

### DESCRIPTION OF DRAWINGS

FIG. 1 is a schematic view of angiotensin II-mediated signaling leading to changes in blood pressure.

FIG. 2 is a schematic view of two distinct rat angiotensin type 1a receptor (ATlaR): ΔE2 vs. E2. CR represents the coding region.

FIG. 3 shows that E2 is less efficiently translated in vitro than ΔE2. Lane 1: negative control; Lane 2: positive control; Lane 3: E2; Lane 4: ΔE2 for the in vitro translation assays.

FIG. 4 shows that ATlaR density is higher in rat aortic smooth muscle cells (A10) transfected with ΔE2 versus E2 plasmid DNA.

FIG. 5 shows that E2m (ATlaR E2 mutant with all four AUGS in the 5′ LS disrupted) is more efficiently translated in vitro than E2.

FIGS. 6A & 6B show that ATlaR density is significantly higher in A10 cells transfected with E2m compared to E2 plasmid DNA.

FIG. 6C shows that Angiotensin II-induced IP production is significantly higher in E2m plasmid DNA transfected cells than that in E2 plasmid DNA transfected cells.

FIG. 7 shows the presence of E2 (E1,2,3) and ΔE2 (E1,3) splicing variants in different tissues. RASMC: rat aortic smooth muscle cells.

FIG. 8 shows the ratio of renal cortex ΔE2/E2 in different strains of rats.

FIG. 9A presents results that show that two splicing variants of At1aR exists in rat renal cortex as shown by real-time PCR using primers specific to E2 and ΔE2 variants with total RNA isolated from the renal cortex of Sprague Dawley (SD) rats.

FIG. 9B shows that the ratio of ΔE2 to total At1aR mRNA is higher in normotensive DS rats than that in SD or Fischer 344/BN rats.

FIG. 10 shows that the ratio of ΔE2 to total AT1aR mRNA is higher in hypertensive DS rats maintained on HS diet than in hypertensive DS rats maintained on NS diet.

FIG. 11 shows representative 21-nucleotide siRNAs. T: AT1R ΔE2 target sequence. S: sense strand. AS: antisense strand.

FIG. 12 shows representative 22-, 23-, 24-, 25-nucleotide siRNAs. T: AT1R ΔE2 target sequence. S: sense strand. AS: antisense strand.

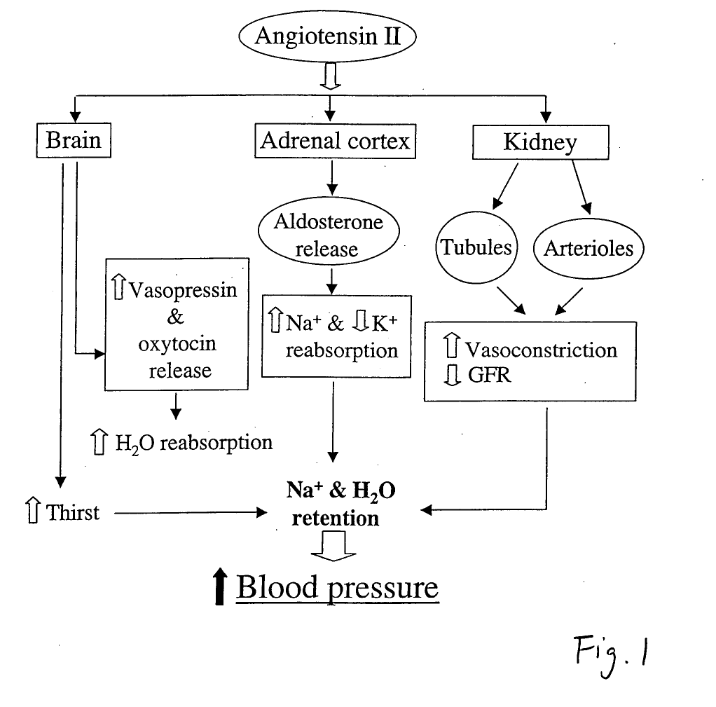

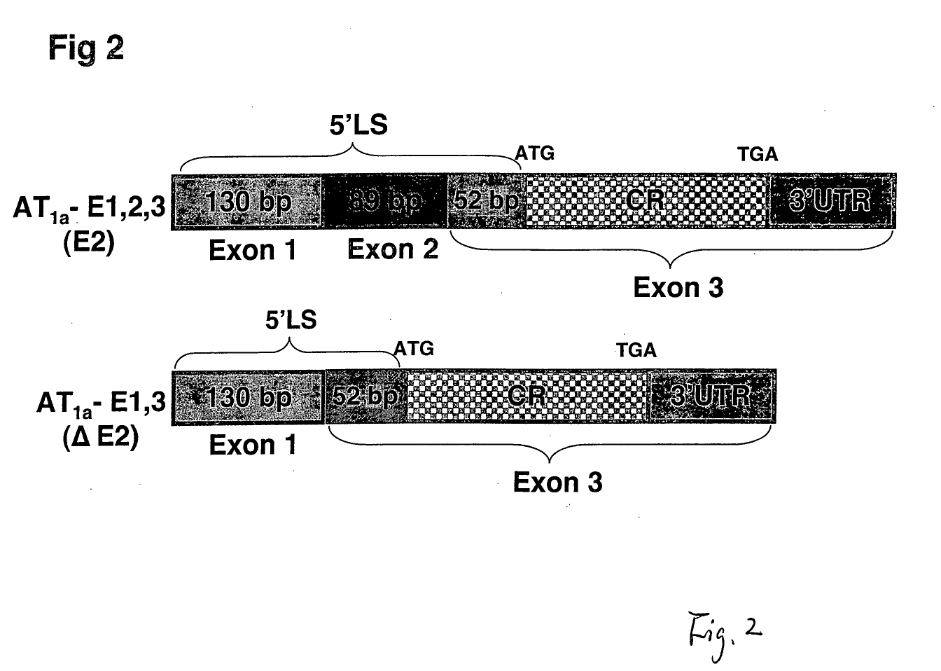

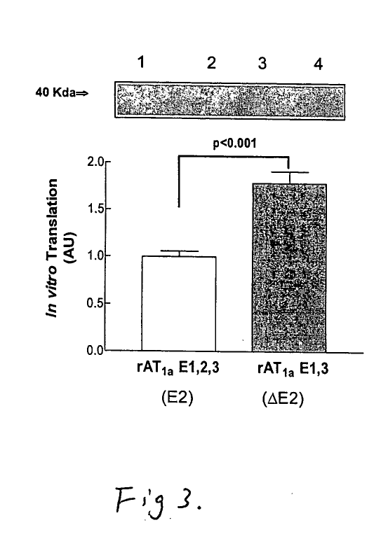

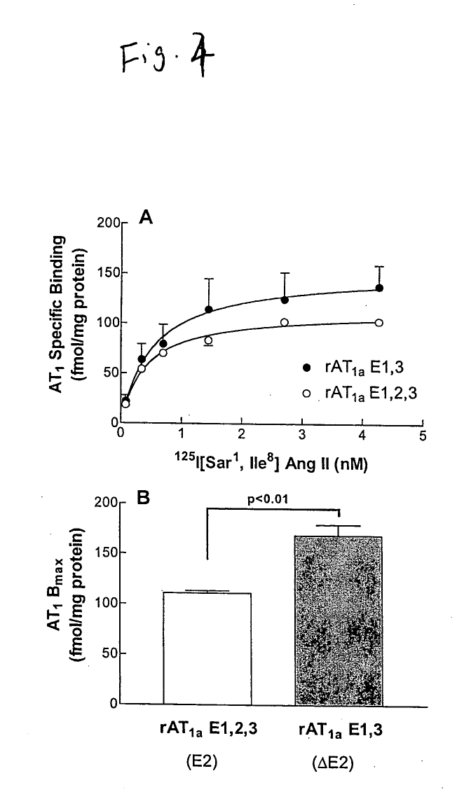

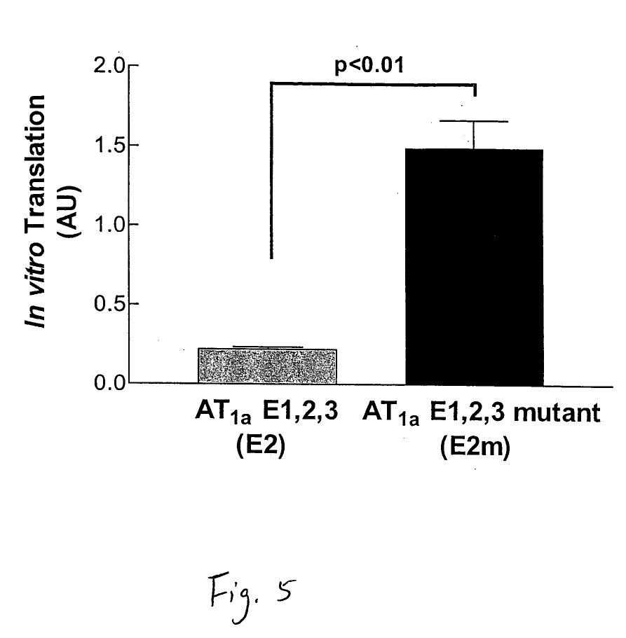

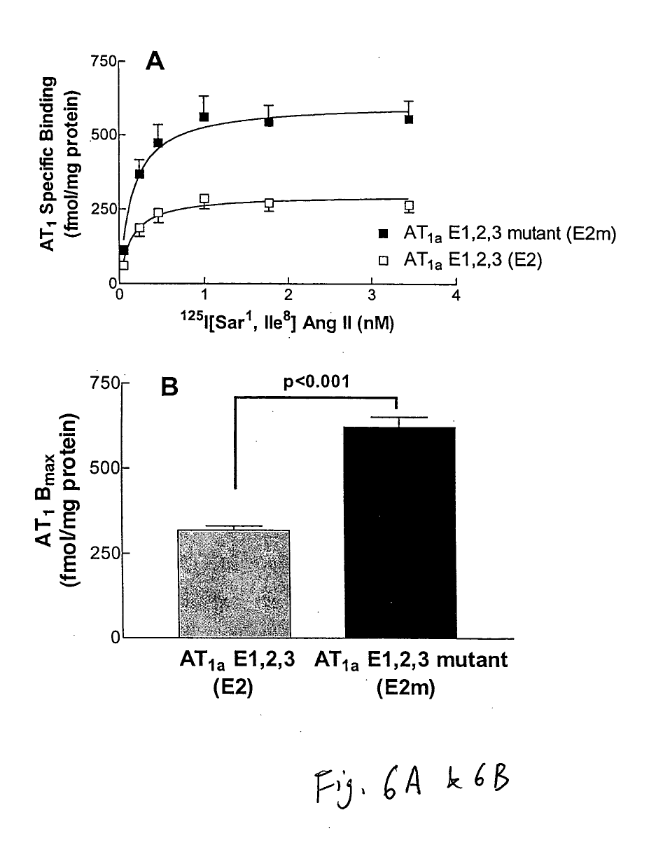

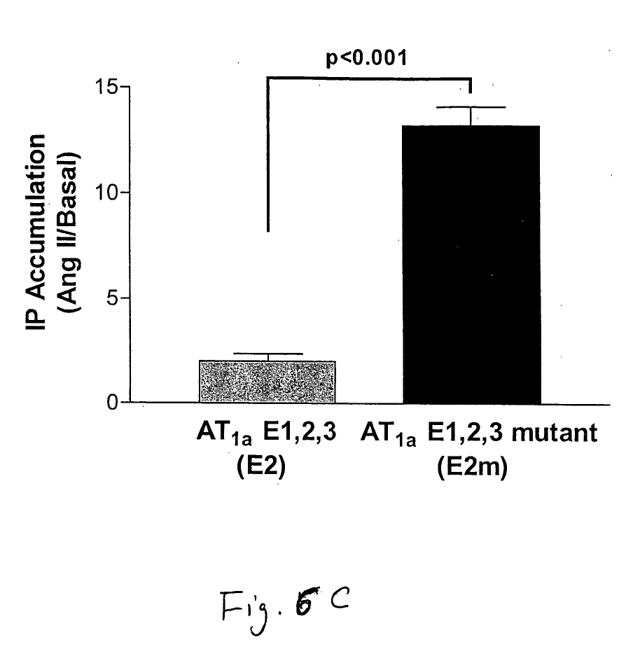

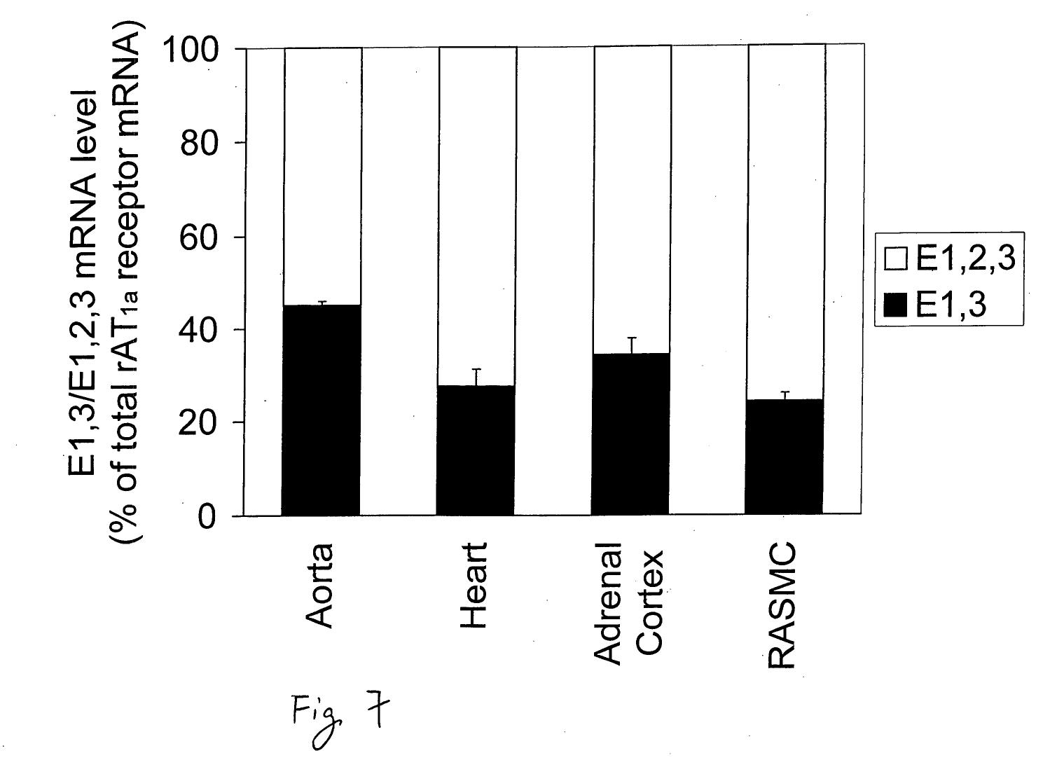

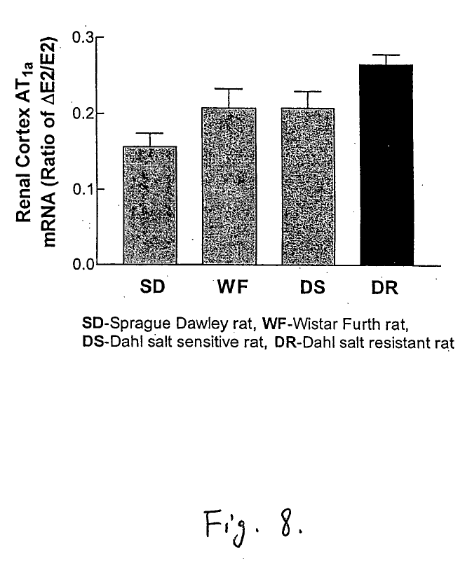

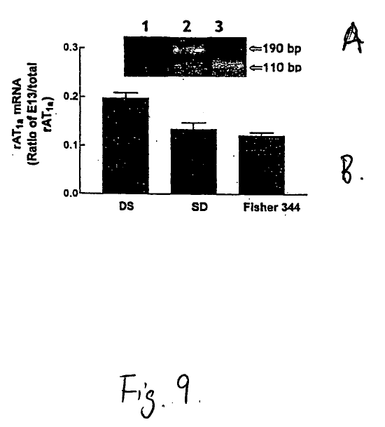

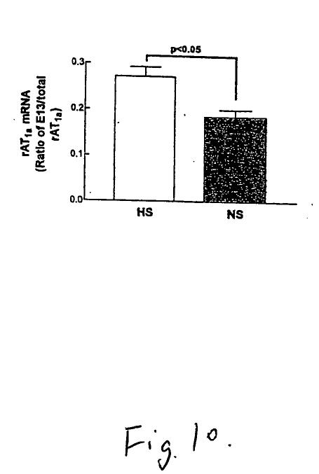

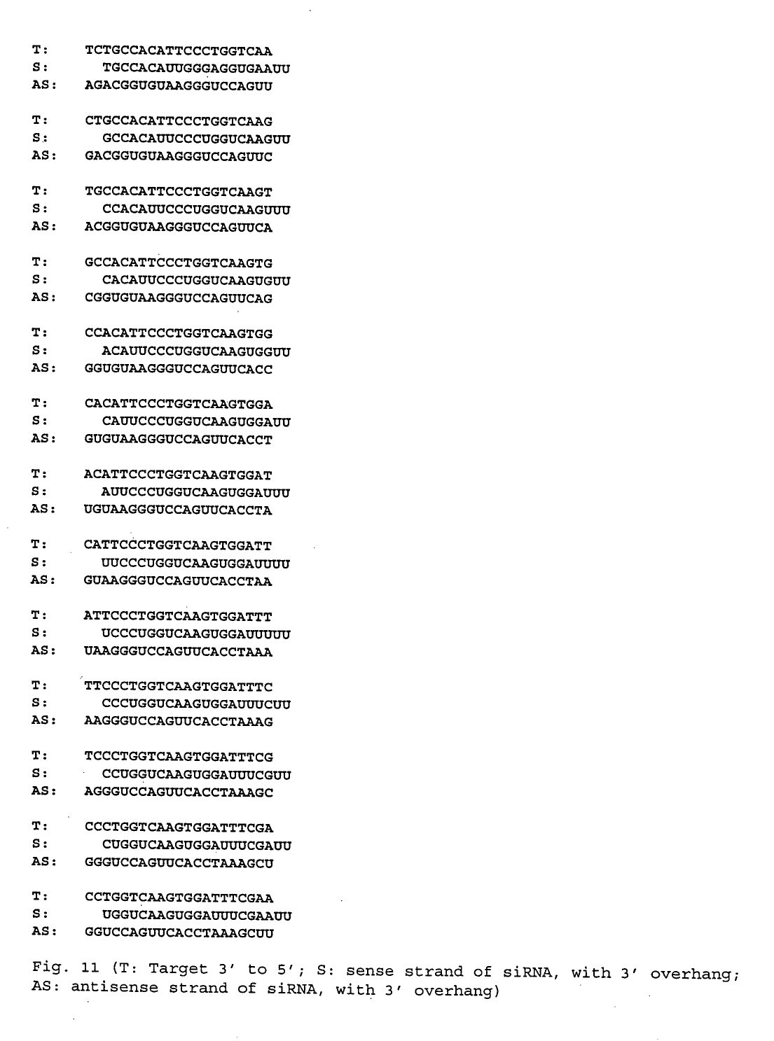

### DETAILED DESCRIPTION OF THE INVENTION

I. Definitions

For convenience, certain terms employed in the specification, examples, and appended claims are collected here.

A “patient” or “subject” to be treated by a disclosed method can mean either a human or non-human animal.

The term “condition,” as used herein, is intended to include active disorders, e.g., disorders which have manifested their symptoms, and predisposition to a disorder (e.g., the genetic tendency toward a disorder which has not yet manifested itself symptomatically).

The term “expression” with respect to a gene sequence refers to transcription of the gene and, as appropriate, translation of the resulting mRNA transcript to a protein. Thus, as will be clear from the context, expression of a protein coding sequence results from transcription and translation of the coding sequence. A method that decreases the expression of a gene may do so in a variety of ways (none of which are mutually exclusive), including, for example, by inhibiting transcription of the gene, decreasing the stability of the mRNA, or decreasing translation of the mRNA. While not wishing to be bound to a particular mechanism, it is generally thought that siRNA techniques decrease gene expression by stimulating the degradation of targeted mRNA species.

As used herein, the term “nucleic acid” refers to polynucleotides such as deoxyribonucleic acid (DNA) and ribonucleic acid (RNA). The term should also be understood to include, as applicable to the embodiment being described, single-stranded (such as sense or antisense) and double-stranded polynucleotides. The “canonical” nucleotides are adenosine (A), guanosine (G), cytosine (C), thymidine (T), and uracil (U), and include a ribose-phosphate backbone, but the term nucleic acid is intended to include polynucleotides comprising only canonical nucleotides as well as polynucleotides including one or more modifications to the sugar phosphate backbone or the nucleoside. DNA and RNA are chemically different because of the absence or presence of a hydroxyl group at the 2′ position on the ribose. Modified nucleic acids that cannot be readily termed DNA or RNA (e.g., in which an entirely different moiety is positioned at the 2′ position) and nucleic acids that do not contain a ribose-based backbone may be referred to as XNAs. Examples of XNAs are peptide nucleic acids (PNAs) in which the backbone is a peptide backbone, and locked nucleic acids (LNAs) containing a methylene linkage between the 2′ and 4′ positions of the ribose. An “unmodified” nucleic acid is a nucleic acid that contains only canonical nucleotides and a DNA or RNA backbone.

“Small molecule” as used herein, is meant to refer to a compound that has a molecular weight of less than about 5 kD and most preferably less than about 2.5 kD. Small molecules can be nucleic acids, peptides, polypeptides, peptidomimetics, carbohydrates, lipids or other organic (carbon containing) or inorganic molecules. Many pharmaceutical companies have extensive libraries of chemical and/or biological mixtures comprising arrays of small molecules, often fungal, bacterial, or algal extracts, which can be screened with any of the assays of the invention.

The term “pharmaceutically acceptable salts” refers to physiologically and pharmaceutically acceptable salts of the compounds of the invention, i.e., salts that retain the desired biological activity of the parent compound and do not impart undesired toxicological effects thereto.

By “recombinant virus” is meant a virus that has been genetically altered, e.g., by the addition or insertion of a heterologous nucleic acid construct into the particle.

As used herein, the term “RNAi construct” is a generic term used throughout the specification to include small interfering RNAs (siRNAs), hairpin RNAs, and other RNA species which can be cleaved in vivo to form siRNAs. RNAi constructs herein also include expression vectors (also referred to as RNAi expression vectors) capable of giving rise to transcripts which form dsRNAs or hairpin RNAs in cells, and/or transcripts which can produce siRNAs in vivo. “RNAi expression vector” (also referred to herein as a “dsRNA-encoding plasmid”) refers to replicable nucleic acid constructs used to express (transcribe) RNA which produces siRNA moieties in the cell in which the construct is expressed. Such vectors include a transcriptional unit comprising an assembly of (1) genetic element(s) having a regulatory role in gene expression, for example, promoters, operators, or enhancers, operatively linked to (2) a “coding“sequence which is transcribed to produce a double-stranded RNA (two RNA moieties that anneal in the cell to form an siRNA, or a single hairpin RNA which can be processed to an siRNA), and (3) appropriate transcription initiation and termination sequences. The choice of promoter and other regulatory elements generally varies according to the intended host cell. In general, expression vectors of utility in recombinant DNA techniques are often in the form of “plasmids“which refer to circular double stranded DNA loops which, in their vector form, are not bound to the chromosome. “Plasmid” and “vector” are used herein interchangeably as the plasmid is the most commonly used form of vector. However, the invention is intended to include other forms of expression vectors that serve equivalent functions and become known in the art subsequently hereto.

In the expression vectors, regulatory elements controlling transcription can be generally derived from mammalian, microbial, viral or insect genes. The ability to replicate in a host, usually conferred by an origin of replication, and a selection gene to facilitate recognition of transformants may additionally be incorporated. Vectors derived from viruses, such as retroviruses, adenoviruses, and the like, may be employed.

The term “small interfering RNAs” or “siRNAs” refers to nucleic acids around 19-30 nucleotides in length, and more preferably 21-23 nucleotides in length. The siRNAs are double-stranded, and may include short overhangs at each end. Preferably, the overhangs are 1-6 nucleotides in length at the 3′ end. It is known in the art that the siRNAs can be chemically synthesized, or derive from a longer double-stranded RNA or a hairpin RNA. The siRNAs have significant sequence similarity to a target RNA so that the siRNAs can pair to the target RNA and result in sequence-specific degradation of the target RNA through an RNA interference mechanism. Optionally, the siRNA molecules comprise a 3′ hydroxyl group. “Hybridization” refers to any process by which a strand of nucleic acid binds with a complementary strand through base pairing. Two single-stranded nucleic acids “hybridize” when they form a double-stranded duplex. The region of double-strandedness can include the full-length of one or both of the single-stranded nucleic acids, or all of one single stranded nucleic acid and a subsequence of the other single stranded nucleic acid, or the region of double-strandedness can include a subsequence of each nucleic acid. Hybridization also includes the formation of duplexes that contain certain mismatches, provided that the two strands are still forming a double stranded helix. “Stringent hybridization conditions” refers to hybridization conditions resulting in essentially specific hybridization.

The term “antisense oligonucleotides” means a sequence of nucleic acids constructed so as to bind to the mRNA encoding a certain protein and thereby prevent translation of the mRNA into protein. Antisense oligonucleotides corresponding to regions of mRNA were synthesized by standard chemical techniques.

As used herein, the terms “transduction” and “transfection” are art-recognized and mean the introduction of a nucleic acid, e.g., an expression vector, into a recipient cell by nucleic acid-mediated gene transfer. “Transformation,” as used herein, refers to a process in which a cell's genotype is changed as a result of the cellular uptake of exogenous DNA or RNA, and, for example, the transformed cell expresses an RNAI construct. A cell has been “stably transfected” with a nucleic acid construct when the nucleic acid construct is capable of being inherited by daughter cells. “Transient transfection” refers to cases where exogenous DNA does not integrate into the genome of a transfected cell, e.g., where episomal DNA is transcribed into mRNA and translated into protein.

II. Screening for Candidate Agents

The candidate agents used in the invention may be pharmacologic agents already known in the art or may be agents previously unknown to have any pharmacological activity. The agents may be naturally arising or designed or prepared in the laboratory. They may be isolated from microorganisms, animals, or plants, or may be produced recombinantly, or synthesized by chemical methods known in the art. In some embodiments, candidate agents are identified from small chemical libraries, peptide libraries, or collections of natural products using the methods of the present invention. Tan et al. described a library with over two million synthetic compounds that is compatible with miniaturized cell-based assays (J. Am. Chem. Soc. 120, 8565-8566, 1998). It is within the scope of the present invention that such a library may be used to screen for agents conferring posttranscriptional regulation on AT1R gene using the methods of the invention. There are numerous commercially available compound libraries, such as the Chembridge DIVERSet. Libraries are also available from academic investigators, such as the Diversity set from the NCI developmental therapeutics program.

One basic approach to search for a subject agent is screening of compound libraries. One may simply acquire, from various commercial sources, small molecule libraries that are believed to meet the basic criteria for useful drugs in an effort to identify useful compounds by “brute force.” Screening of such libraries, including combinatorially generated libraries, is a rapid and efficient way to screen a large number of related (and unrelated) compounds for activity. Combinatorial approaches also lend themselves to rapid evolution of potential drugs by the creation of second, third, and fourth generation compounds modeled on active but otherwise undesirable compounds. It will be understood that undesirable compounds include compounds that are typically toxic, but have been modified to reduce the toxicity or compounds that typically have little effect with minimal toxicity and are used in combination with another compound to produce the desired effect.

On the other hand, many useful pharmacological compounds are compounds structurally related to compounds that interact naturally with the targets, which may be the pre-splicing AT1R mRNA, the E2 or ΔE2 transcript, or the RNABPs. Creating and examining the action of such molecules is known as “rational drug design,” and include making predictions relating to the structure of the targets. Thus, it is understood that a subject agent identified by the present invention may be a small molecule inhibitor or any other compound (e.g., polypeptide or polynucleotide) that may be designed through rational drug design starting from known inhibitors of the targets.

The goal of rational drug design is to produce structural analogs of biologically active target compounds. By creating such analogs, it is possible to fashion drugs that are more active or stable than the natural molecules, have different susceptibility to alteration or may affect the function of various other molecules. In one approach, one can generate a three-dimensional structure for molecules like the targets, and then design a molecule for its ability to interact with the targets. Alternatively, one could design a partially functional fragment of the targets (for example, binding, but not affecting the activity of the RNABPs), thereby creating a competitive inhibitor. This could be accomplished by X-ray crystallography, computer modeling, or by a combination of both approaches.

In a particularly preferred embodiment, rational drug design is directed to synthesizing siRNAs that selectively inhibit the expression of ΔE2. The selective inhibition can be based on the unique juncture of RNA present in the ΔE2 transcript but absent from E2.

It is also possible to use antibodies to inform the structure of a target compound or inhibitor. In principle, this approach yields a pharmacore upon which subsequent drug design can be based. It is possible to bypass protein crystallography altogether by generating anti-idiotype antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

Other suitable inhibitors include antisense molecules, ribozymes, and antibodies (including single chain antibodies).

It will, of course, be understood that all of the screening methods of the present invention are useful in themselves nonwithstanding the fact that effective candidates may not be found in a particular iteration of the screen. The invention provides methods of screening for such candidates, whether or not an active compound is actually identified.

a. siRNA Technology

RNA interference (RNAi) is a phenomenon describing double-stranded (ds)RNA-dependent gene specific posttranscriptional silencing. Initial attempts to harness this phenomenon for experimental manipulation of mammalian cells were foiled by a robust and nonspecific antiviral defense mechanism activated in response to long dsRNA molecules. Gil et al. Apoptosis 2000, 5:107-114. The field was significantly advanced upon the demonstration that synthetic duplexes of 21 nucleotide RNAs could mediate gene specific RNAi in mammalian cells, without invoking generic antiviral defense mechanisms. Elbashir et al. Nature 2001, 411:494-498; Caplen et al. Proc Natl Acad Sci 2001, 98:9742-9747. As a result, small-interfering RNAs (siRNAs) have become powerful tools to dissect gene function. The chemical synthesis of small RNAs is one avenue that has produced promising results. Numerous groups have also sought the development of DNA-based vectors capable of generating such siRNA within cells. Several groups have recently attained this goal and published similar strategies that, in general, involve transcription of short hairpin (sh)RNAs that are efficiently processed to form siRNAs within cells. Paddison et al. PNAS 2002, 99:1443-1448; Paddison et al. Genes & Dev 2002, 16:948-958; Sui et al. PNAS 2002, 8:5515-5520; and Brummelkamp et al. Science 2002, 296:550-553. These reports describe methods to generate siRNAs capable of specifically targeting numerous endogenously and exogenously expressed genes.

Methods of chemically modifying RNA molecules can be adapted for modifying RNAi constructs (see, for example, Heidenreich et al. (1997) Nucleic Acids Res, 25:776-780; Wilson et al. (1994) J Mol Recog 7:89-98; Chen et al. (1995) Nucleic Acids Res 23:2661-2668; Hirschbein et al. (1997) Antisense Nucleic Acid Drug Dev 7:55-61). Merely to illustrate, the backbone of an RNAi construct can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides, a-configuration). Additional modified nucleotides are as follows (this list contains forms that are modified on either the backbone or the nucleoside or both, and is not intended to be all-inclusive): 2′-O-Methyl-2-aminoadenosine; 2′-O-Methyl-5-methyluridine; 2′-O-Methyladenosine; 2′-O-Methylcytidine; 2′-O-Methylguanosine; 2′-O-Methyluridine; 2-Amino-2′-deoxyadenosine; 2-Aminoadenosine; 2-Aminopurine-2′-deoxyriboside; 4-Thiothymidine; 4-Thiouridine; 5-Methyl-2′-deoxycytidine; 5-Methylcytidine; 5-Methyluridine; 5-Propynyl-2′-deoxycytidine; 5-Propynyl-2′-deoxyuridine; Ni-Methyladenosine; Ni-Methylguanosine; N2-Methyl-2′-deoxyguanosine; N6-Methyl-2′-deoxyadenosine; N6-Methyladenosine; O6-Methyl-2′-deoxyguanosine; and 06-Methylguanosine.

The double-stranded structure may be formed by a single self-complementary nucleic acid strand or two complementary nucleic acid strands. Duplex formation may be initiated either inside or outside the cell. The RNAi construct may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications. Given the greater uptake of the modified RNAi nucleic acids disclosed herein, it is understood that lower dosing may be employed than is generally used with traditional RNAI constructs. Inhibition is sequence-specific in that nucleotide sequences corresponding to the duplex region of the RNA are targeted for genetic inhibition.

In certain embodiments, the subject RNAi constructs are “small interfering RNAs” or “siRNAs.” These nucleic acids include an antisense RNA strand that is around 19-30 nucleotides in length, and even more preferably 21-23 nucleotides in length, e.g., corresponding in length to the fragments generated by nuclease “dicing” of long double-stranded RNAs. siRNAs may include a sense strand that is RNA, DNA or XNA. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA antisense molecules comprise a 3′ hydroxyl group. Optionally, the sense strand comprises at least 50%, 60%, 70%, 80%, 90% or 100% modified nucleic acids, while the antisense strand is unmodified RNA. Optionally, the sense strand comprises 100% modified nucleic acids (e.g. DNA or RNA with a phosphorothioate modification at every possible position) while the antisense strand is an RNA strand comprising no modified nucleic acids or no more than 10%, 20%, 30%, 40% or 50% modified RNA nucleic acids.

The siRNA molecules of the present invention can be obtained using a number of techniques known to those of skill in the art. For example, the siRNA can be chemically synthesized or recombinantly produced using methods known in the art. For example, short sense and antisense RNA, DNA or XNA oligomers can be synthesized and annealed to form double-stranded structures with 2-nucleotide overhangs at each end (Caplen, et al. (2001) Proc Natl Acad Sci USA, 98:9742-9747; Elbashir, et al. (2001) EMBO J, 20:6877-88). These double-stranded siRNA structures can then be introduced into cells, either by passive uptake or a delivery system of choice.

In certain embodiments, an RNAi construct is in the form of a hairpin structure. The hairpin can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci U S A, 2002, 99:6047-52). Preferably, such hairpin RNAs are engineered in cells or in an animal to ensure continuous and stable suppression of a desired gene. It is known in the art that siRNAs can be produced by processing a hairpin RNA in the cell. A hairpin may be chemically synthesized such that a sense strand comprises RNA, DNA or XNA, while the antisense strand comprises RNA. In such an embodiment, the single strand portion connecting the sense and antisense portions should be designed so as to be cleavable by nucleases in vivo, and any duplex portion should be susceptible to processing by nucleases such as Dicer.

The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, or affinity purification with antibody can be used to purify siRNAs.

In certain preferred embodiments, at least one strand of the siRNA molecules has a 3′overhang from about 1 to about 6 nucleotides in length, or from 2 to 4 nucleotides in length. More preferably, the 3′ overhangs are 1-3 nucleotides in length. In certain embodiments, one strand has a 3′ overhang and the other strand is blunt-ended or also has an overhang. The length of the overhangs may be the same or different for each strand. In order to further enhance the stability of the siRNA, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythyinidine is tolerated and does not affect the efficiency of RNAi. The absence of a 2′ hydroxyl significantly enhances the nuclease resistance of the overhang in tissue culture medium and may be beneficial in vivo.

An example of siRNAs targeting the RNA juncture between exons1 and 3 unique to the rat ΔE2 has the nucleotide sequence of 5′ -CUGGUCAAGUGGAUUUCGAUU-3′ (with the 3′ overhang of UU) and its complementary strand has the nucleotide sequence of 3′-GGGACCAGUUCACCUAAAGCU-5′ (with the 3′ overhang of GG). As is known in the art, siRNAs having different nucleotide sequences can be made, and these siRNAs can be screened based on their ability to inhibit the target gene expression. In preferred embodiments, however, the RNA juncture between exons1 and 3 unique to ΔE2 naturally limit the number of possible desired siRNAs. As is known in the art, siRNAs targeting the RNA juncture between exons 1 and 3 unique to other mammalian ΔE2s, e.g., human ΔE2, can be readily made, based on the published E2 and ΔE2 nucleic acid sequences.

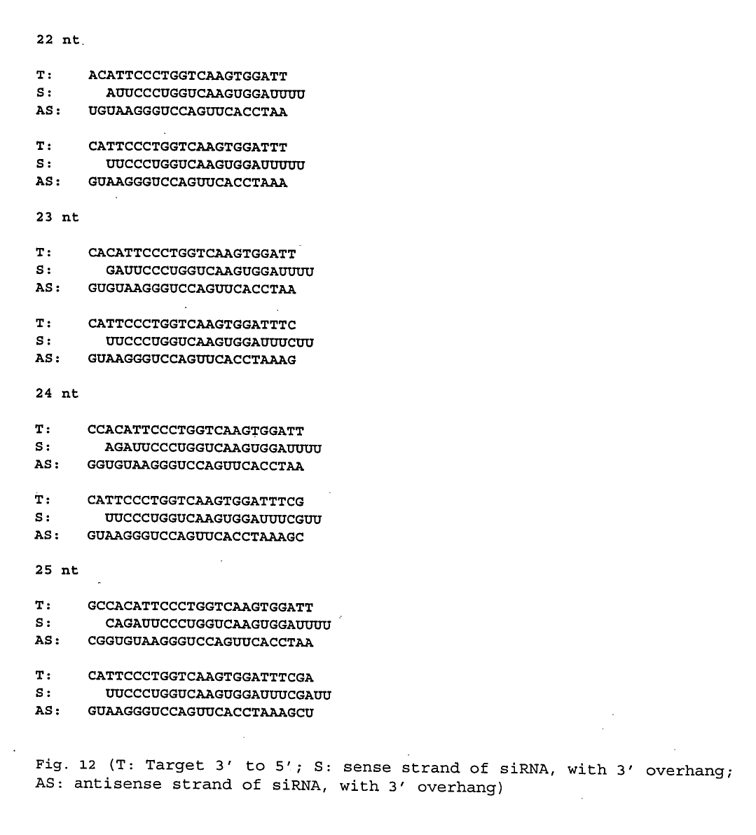

Examples of siRNAs designed to target the RNA juncture between exonsI and 3 unique to the rat ΔE2 are also shown in FIG. 11 and FIG. 12. As is known in the art, siRNAs of nucleotide sequences homologous to these representative sequences can also be used to attenuate the expression of ΔE2 transcript. Alternatively, siRNAs that can specifically hybridize with the target sequences are also contemplated in the present invention. Hybridization can be carried out under wash conditions of 2×SSC at 22° C., and more preferably 0.2×SSC at 65° C., to a target sequence.

As is known in the art, siRNAs generally tolerate mutations in the 5′ end, while the 3′ end exhibited low tolerance (Amarzguioui et al., Nucleic Acids Res. 31(2): 589-95, January 2003). Accordingly, the preferred siRNAs in the present invention are designed to complement the 5′end of a target sequence, whereas mismatches to the 3′ end of a target sequence can be tolerated. The tolerance to mismatches at the 5′ end of siRNAs, i.e., the 3′ end of target sequences, is especially useful, as single nucleotide polymorphism of AT1R gene is known in the art to be present.

The RNAi constructs of the invention may also be admixed, encapsulated, conjugated or otherwise associated with other molecules, molecule structures or mixtures of compounds, as for example, liposomes, polymers, receptor targeted molecules, oral, rectal, topical or other formulations, for assisting in uptake, distribution and/or absorption. The subject RNAi constructs can be provided in formulations also including penetration enhancers, carrier compounds and/or transfection agents.

Representative United States patents that teach the preparation of such uptake, distribution and/or absorption assisting formulations which can be adapted for delivery of RNAi constructs, particularly siRNA molecules, include, but are not limited to, U.S. Pat. Nos. 5,108,921; 5,354,844; 5,416,016; 5,459,127; 5,521,291; 51543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756.

The RNAi constructs of the invention also encompass any pharmaceutically acceptable salts, esters or salts of such esters, or any other compound which, upon administration to an animal including a human, is capable of providing (directly or indirectly) the biologically active metabolite or residue thereof. Accordingly, for example, the disclosure is also drawn to RNAi constructs and pharmaceutically acceptable salts of the siRNAs, pharmaceutically acceptable salts of such RNAi constructs, and other bioequivalents.

b. Antisense Technology

Another aspect of the invention relates to the use of the isolated nucleic acid in “antisense” therapy. As used herein, “antisense” therapy refers to administration or in situ generation of oligonucleotide probes or their derivatives which specifically hybridize (e.g. bind) under cellular conditions, with the cellular mRNA and/or genomic DNA encoding a target splicing variant of AT1R, preferably ΔE2, so as to inhibit translation of the target splicing variant. The binding may be by conventional base pair complementarity, or, for example, in the case of binding to DNA duplexes, through specific interactions in the major groove of the double helix. In general, “antisense” therapy refers to the range of techniques generally employed in the art, and includes any therapy which relies on specific binding to oligonucleotide sequences.

An antisense molecule of the present invention can be delivered, for example, as an expression plasmid which, when transcribed in the cell, produces RNA which is complementary to at least a unique portion of a target splicing variant of AT1R, preferably ΔE2. Alternatively, the antisense molecule is an oligonucleotide probe which is generated ex vivo and which, when introduced into the cell, causes inhibition of expression by hybridizing with a target splicing variant of AT1R and/or genomic sequences of an AT1R gene. Such oligonucleotide probes are preferably modified oligonucleotides which are resistant to endogenous nucleases, e.g., exonucleases and/or endonucleases, and are therefore stable in vivo. Exemplary nucleic acid molecules for use as antisense oligonucleotides are phosphoramidite, phosphorothioate, and methylphosphonate analogs of DNA (see also U.S. Pat. Nos. 5,176,996; 5,264,564; and 5,256,775), or peptide nucleic acids (PNAs). Additionally, general approaches to constructing oligomers useful in antisense therapy have been reviewed, for example, by Van der Krol et al. (1988) Biotechniques 6:958-976; and Stein et al. (1988) Cancer Res 48:2659-2668.

Accordingly, the agents, e.g., RNAI molecules, of the invention are useful in therapeutic, diagnostic, and research contexts. In therapeutic applications, the agents are utilized in a manner appropriate for antisense therapy in general. For such therapy, the agents of the invention can be formulated for a variety of routes of administration, including systemic and topical or localized administration. Techniques and formulations generally may be found in Remington's Pharmaceutical Sciences, Meade Publishing Co., Easton, Pa. For systemic administration, injection is preferred, including intramuscular, intravenous, intraperitoneal, and subcutaneous. For injection, the agents of the invention can be formulated in liquid solutions, preferably in physiologically compatible buffers such as Hank's solution or Ringer's solution. In addition, the agents may be formulated in solid form and redissolved or suspended immediately prior to use. Lyophilized forms are also included.

Systemic administration can also be by transmucosal or transdermal means, or the compounds can be administered orally. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For oral administration, the agents are formulated into conventional oral administration forms such as capsules, tablets, and tonics. For topical administration, the agents of the invention are formulated into ointments, salves, gels, or creams as is generally known in the art.

In addition to use in therapy, the agents of the invention may be used as diagnostic reagents to detect the presence or absence of the target splicing variant (E2 or ΔE2) sequences to which they specifically bind.

Likewise, the antisense molecules of the present invention, by antagonizing the normal biological activity of a target splicing variant (E2 or ΔE2), e.g., by reducing the level of its translation and/or expression, can be used in the manipulation of tissue, e.g. tissue maintenance, differentiation or growth, both in vivo and ex vivo.

Furthermore, the anti-sense techniques (e.g., microinjection of antisense molecules, or transfection with plasmids whose transcripts are anti-sense with regard to a target splicing variant or gene sequence) can be used to investigate the role of alternative splicing in regulating functions of cells where such alternative splicing exists, e.g., aorta, renal cortex, aortic smooth muscles. Such techniques can be utilized in cell culture, but can also be used in the creation of transgenic animals (described infra).

c. Combinatorial Libraries

Compounds suitable for use in the present invention, particularly libraries of variants having various representative classes of substituents, are amenable to combinatorial chemistry and other parallel synthesis schemes (see, for example, PCT WO 94/08051). The result is that large libraries of related compounds, e.g., a variegated library of compounds represented above, can be screened rapidly in high throughput assays in order to identify potential lead compounds, as well as to refine the specificity, toxicity, and/or cytotoxic-kinetic profile of a lead compound. For instance, in vitro translation assays using E2 and ΔE2 can be used to screen a library of the subject compounds for those inhibit the translation of ΔE2 or promote the translation of E2 or both.

Simply for illustration, a combinatorial library for the purposes of the present invention is a mixture of chemically related compounds which may be screened together for a desired property. The preparation of many related compounds in a single reaction greatly reduces and simplifies the number of screening processes which need to be carried out. Screening for the appropriate physical properties can be done by conventional methods.

Diversity in the library can be created at a variety of different levels. For instance, the substrate aryl groups used in the combinatorial reactions can be diverse in terms of the core aryl moiety, e.g., a variegation in terms of the ring structure, and/or can be varied with respect to the other substituents.

A variety of techniques are available in the art for generating combinatorial libraries of small organic molecules to be screened by the methods of the present invention. (See, for example, Blondelle et al. (1995) Trends Anal. Chem. 14:83; the Affymax U.S. Pat. 5,359,115 and 5,362,899; the Ellman U.S. Pat. No. 5,288,514; the Still et al. PCT publication WO 94/08051; the ArQule U.S. Pat. No. 5,736,412 and 5,712,171; Chen et al. (1994) JACS 116:2661; Kerr et al. (1993) JACS 115:252; PCT publications WO92/10092, WO93/09668 and WO91/07087; and the Lerner et al. PCT publication WO93/20242, all incorporated herein by reference in full). Accordingly, a variety of libraries on the order of about 100 to 1,000,000 or more diversomers of the candidate agents can be synthesized and screened for particular activity or property.

In an exemplary embodiment, a library of candidate agent diversomers can be synthesized utilizing a scheme adapted to the techniques described in the Still et al., PCT publication WO 94/08051 (incorporated herein in full), e.g., being linked to a polymer bead by a hydrolyzable or photolyzable group, optionally located at one of the positions of the candidate antagonists or a substituent of a synthetic intermediate. According to the Still et al. technique, the library is synthesized on a set of beads, each bead including a set of tags identifying the particular diversomer on that bead. The bead library can then be mixed with translation reaction mixtures, e.g., in an in vitro translation assay.

Many variations on the above and related pathways permit the synthesis of widely diverse libraries of compounds which may be tested as the subject agents.

d. Screening Assays

There are a variety of assays available for determining the ability of a compound to regulate AT1R mRNA post-transcriptionally that can be performed in high-throughput formats. A compound may inhibit the alternative splicing that specifically results in ΔE2, or promote the alternative splicing that specifically results in E2, or do both. Another type of compound of the present invention selectively inhibits translation of ΔE2, or promotes translation initiation from E2 without affecting the cis-regulatory effect of E2 itself, or does both. A third type of compound of the present invention modulates the activities of RNABPs that interact with the 5′ LS of AT1R transcripts.

A quick, inexpensive and easy assay to run is a binding assay. Binding of a molecule to a target (e.g., AT1R pre-spliced mRNA, E2 or ΔE2 transcripts) may, in and of itself, be inhibitory, due to steric, allosteric or charge-charge interactions. This can be performed in solution or on a solid phase and can be utilized as a first round screen to rapidly eliminate certain compounds before moving into more sophisticated assays. In one embodiment of this kind, the screening of compounds that bind to AT1R pre-spliced mRNA, E2 or ΔE2 transcripts, or RNABPs, or biologically active peptide fragment(s) of RNABPs is provided. In certain embodiments, a biologically active peptide fragment can be a domain that is responsible for RNABPs' interaction with the 5′ LS.

The target AT1R pre-spliced mRNA, E2 or ΔE2 transcripts, or RNABPs (“the targets”) may be either free in solution, fixed to a support, expressed in a cell. Either the targets or the compounds subject to screening may be labeled, thereby permitting determining of binding. In another embodiment, the assay may measure the inhibition of binding of the targets to a natural or artificial substrate or binding partner. Competitive binding assays can be performed in which one of the agents included in the assay is labeled. Usually, the targets will be the labeled species, decreasing the chance that the labeling will interfere with the binding moiety's function. One may measure the amount of free label versus bound label to determine binding or inhibition of binding.

In certain embodiments, an in vitro translation assay may be followed by the binding assay as described above to further test the selected compound's ability to regulate AT1R mRNA post-transcriptionally. Many in vitro translation assay techniques are well known in the art. General aspects of in vitro translation assays are described in U.S. Pat. Nos. 4,668,624 and 5,434,079, which are incorporated by reference herein in full. These references describe in vitro translation assays conducted for producing proteins other than AT1Rs in the present invention. Many of the components of the in vitro translation assays can nevertheless be modified according to principles well understood in the art for use in the assays for identifying the candidate agents of the present invention.

In certain embodiments, in vitro splicing assays are conducted. AT1R gene is used as a template in combination with a reporter gene, for example, luciferase, and candidate agents will be screened for their ability to decrease the amount of exon 2 removed by splicing, i.e., resulting in an increase of E2 variants or decrease of ΔE2 variant or both. Alternatively, part of the AT1R gene can be used as a template in the in vitro splicing assay.

In addition to cell-free assays, compounds can also be tested in cell-based assays. Various cell lines can be utilized for screening of the candidate agents, e.g., the rat aortic smooth muscle cell line A10. As is known in the art, cell lines expressing AT1Rs can be created via transfections with nucleic acids encoding AT1R.

Depending on the assay, culture may be required. The cell may then be examined by virtue of a number of different physiologic assays, e.g., reporter gene activities, assays indicating AT1R expression levels such as measuring angiotensin II-induced inositol phosphate production and/or AT1R-specific binding by angiotensin II.

In one embodiment, cells which express AT1R can be contacted with a test compound/agent of interest appropriately formulated based on its biochemical nature, with the assay scoring for, e.g., the density of AT1R expressed on the surface of the cells in the presence of the test compound/agent.

The present invention also contemplates the use of various animal models. Transgenic animals may be created with constructs that permit ectopic AT1R expression, and activity of the AT1R to be controlled and monitored. The generation of these animals can be based on techniques well known in the art. In a preferred embodiment, hypertensive animal models such as DS rats can be used, to screen for test compounds that can maintain or decrease glomerular AT1R density in response to high salt diet.

e. Transgenic Animals:

The present invention is further directed to a transgenic non-human eukaryotic animal, preferably a rodent, such as a mouse or other animal capable of developing detectable characteristics from the expression of an RNAi molecule of the present invention. The RNAi molecule is introduced into the animal, or an ancestor of the animal, at an embryonic stage, preferably the one cell, or fertilized oocyte stage and generally not later than about the 8-cell stage. The zygote or embryo is then developed to term in a pseudo-pregnant foster female. The plasmid DNA is introduced into an animal embryo so as to be chromosomally incorporated in a state which results in super-endogenous expression of its corresponding RNA molecule, e.g., an siRNA targeting ΔE2, an antisenese RNA targeting ΔE2, an RNA encoding E2 or ΔE2.

Transgenic mammals are prepared in a number of ways. A transgenic organism is one that has an extra or exogenous fragment of DNA in its genome. In order to achieve stable inheritance of the extra or exogenous DNA fragment, the integration event must occur in a cell type that can give rise to functional germ cells, either sperm or oocytes. Two animal cell types that can form germ cells and into which DNA can be introduced readily are fertilized egg cells and embryonic stem cells. Embryonic stem (ES) cells can be returned from in vitro culture to a “host” embryo where they become incorporated into the developing animal and can give rise to transgenic cells in all tissues, including germ cells. The ES cells are transfected in culture and then the mutation is transmitted into the germline by injecting the cells into an embryo. The animals carrying mutated germ cells are then bred to produce transgenic offspring.

A preferred method for making the subject transgenic animals is by zygote injection. This method is described, for example in U.S. Pat. No. 4,736,866. The method involves injecting DNA into a fertilized egg, or zygote, and then allowing the egg to develop in a pseudo-pregnant mother. The zygote can be obtained using male and female animals of the same strain or from male and female animals of different strains. The transgenic animal that is born is called a founder, and it is bred to produce more animals with the same DNA insertion. In this method of making transgenic animals, the new DNA typically randomly integrates into the genome by a non-homologous recombination event. One to many thousands of copies of the DNA may integrate at one site in the genome.

In a preferred embodiment, a vector (e.g., a retroviral vector) comprising a desired transgene (e.g., encoding an RNAi molecule of the present invention) is introduced to an oocyte by microinjection. As is known in the art, the retroviral vector comprising the transgene will be integrated into the oocyte genome and the transgene will be expressed.

In a preferred embodiment, the transgene comprises a hairpin RNA of the present invention. In a preferred embodiment, the hairpin RNA targets the juncture between exons 1 and 3 of AT1R. In a preferred embodiment, the hairpin RNA expression is driven by a single promoter.

In another preferred embodiment, the transgene comprises a double-strand siRNA. In a preferred embodiment, the siRNA targets the juncture between exons 1 and 3 of AT1R. In a preferred embodiment, the siRNA expression is driven by two promoters from opposite directions such that both strands of the siRNA will be expressed.

Generally, the DNA comprising a transgene is injected into one of the pronuclei, usually the larger male pronucleus. The zygotes are then either transferred the same day, or cultured overnight to form 2-cell embryos and then transferred into the oviducts of the pseudo-pregnant females. The animals born are screened for the presence of the desired integrated DNA. By a pseudo-pregnant female is intended a female in estrous who has mated with a vasectomized male; she is competent to receive embryos but does not contain any fertilized eggs. Pseudo-pregnant females are important for making transgenic animals since they serve as the surrogate mothers for embryos that have been injected with DNA or embryonic stem cells.

Putative founders are screened for the presence of the transgene by PCR analysis of tail DNA as described in the Example 6. Transgene expression can be initially evaluated by RNA analysis using the Northern blot technique. Preferably, the ratio of E2 to ΔE2 transcripts or total AT1R mRNA is increased in renal tissue of transgenic but not control mice. To ascertain the expression of AT1R at the cell surface, binding experiments can be performed using transgenic and control glomeruli. To this end, labeled angiotensin II radioligand can be employed.

The founder animals can be used to produce stable lines of transgenic animals that superexpress the RNAi as desired. For ease of propagation, male founder mice are preferred. The animals are observed clinically. Analyses of transgene copy number (to exclude multiple transgene insertion sites), total mRNA expression, ratio of the splicing variants and protein expression in these animals are also performed. These studies may provide information about the age of onset of illness, the duration of illness, the penetrance of the phenotype, the range of pathologic findings and the dependence of phenotype upon levels of protein expression.

The present invention also contemplates creating transgenic animals by homologous recombination. The term “homologous recombination” refers to the process of DNA recombination based on sequence homology of nucleic acid sequences in a construct with those of a target sequence, such as a target allele, in a genome or DNA preparation. Accordingly, the nucleic acid sequences present in the construct are identical or highly homologous, that is, they are more than 60%, preferably more than 70%, highly preferably more than 80%, and most preferably more than 90% sequence identity to a target sequence located within a cell genome. In a particular embodiment, the homologous recombination vector has 95%-98% sequence identity to a target sequence located within a cell genome.

In a preferred embodiment, the subject invention provides a construct which, by homologous recombination with a genomic DNA, alters the level of AT1R splicing variants present in the cells.

In preferred embodiments, the nucleotide sequence used as the construct can be comprised of (1) DNA from some portion of the endogenous AT1R gene (exon sequence, intron sequence, promoter sequences, etc.) which direct recombination and (2) heterologous transcriptional regulatory sequence(s) which is to be operably linked to the coding sequence for the genomic AT1R gene upon recombination of the construct. For use in generating cultures of AT1R producing cells, the construct may further include a reporter gene to detect the presence of the knockout construct in the cell.

The construct is inserted into a cell, and integrates with the genomic DNA of the cell in such a position so as to provide the heterologous regulatory sequences in operative association with the native AT1R gene. Such insertion occurs by homologous recombination, i.e., recombination regions of the construct that are homologous to the endogenous AT1R gene sequence hybridize to the genomic DNA and recombine with the genomic sequences so that the construct is incorporated into the corresponding position of the genomic DNA.

The terms “recombination region” or “targeting sequence” refer to a segment (i.e., a portion) of a construct having a sequence that is substantially identical to or substantially complementary to a genomic gene sequence, e.g., including 5′ flanking sequences of the genomic gene, and can facilitate homologous recombination between the genomic sequence and the targeting transgene construct.

As used herein, the term “replacement region” refers to a portion of a construct which becomes integrated into an endogenous chromosomal location following homologous recombination between a recombination region and a genomic sequence. In a preferred embodiment, the replacement region of the present invention encodes E2. In a particularly preferred embodiment, the E2 of the replacement region is not subject to alternative splicing that removes exon 2 in the transgenic animal. In another aspect of the invention, the replacement region encodes ΔE2, in situations, e.g. where higher expression of AT1R may be desirable.

The heterologous regulatory sequences, e.g., which are provided in the replacement region, can include one or more of a variety elements, including: promoters (such as constitutive or inducible promoters), enhancers, negative regulatory elements, locus control regions, transcription factor binding sites, or combinations thereof. Promoters/enhancers which may be used to control the expression of the targeted gene in vivo include, but are not limited to, the cytomegalovirus (CMV) promoter/enhancer (Karasuyama et al., 1989, J. Exp. Med., 169:13), the human β-actin promoter (Gunning et al. (1987) PNAS 84:4831-4835), the glucocorticoid-inducible promoter present in the mouse mammary tumor virus long terminal repeat (MMTV LTR) (Klessig et al. (1984) Mol. Cell Biol. 4:1354-1362), the long terminal repeat sequences of Moloney murine leukemia virus (MuLV LTR) (Weiss et al. (1985) RNA Tumor Viruses, Cold Spring Harbor Laboratory, Cold Spring Harbor, N.Y.), the SV40 early or late region promoter (Bemoist et al. (1981) Nature 290:304-310; Templeton et al. (1984) Mol. Cell Biol., 4:817; and Sprague et al. (1983) J. Virol., 45:773), the promoter contained in the 3′ long terminal repeat of Rous sarcoma virus (RSV) (Yamamoto et al., 1980, Cell, 22:787-797), the herpes simplex virus (HSV) thymidine kinase promoter/enhancer (Wagner et al. (1981) PNAS 82:3567-71), and the herpes simplex virus LAT promoter (Wolfe et al. (1992) Nature Genetics, 1:379-384).

In still other embodiments, the replacement region merely deletes a negative transcriptional control element of the native AT1R gene, e.g., to activate expression, or ablates a positive control element, e.g., to inhibit expression of the targeted gene.

The animals of the present invention can be used as tester animals for materials of interest, e.g. agents to prevent and/or treat angiotensin II-related diseases. An animal is treated with the material of interest, and a reduced incidence or delayed onset of such a disease, e.g., hypertension, as compared to untreated animals, is detected as an indication of protection and/or response to treatment.

The animals of the invention may also be used as models for the molecular mechanism of angiotensin II-related diseases.

f. Gene Therapy:

This invention also provides expression vectors containing a nucleic acid encoding an RNAi of the present invention, e.g., siRNAs targeting the juncture between exons 1 and 3 of AT1R, operably linked to at least one transcriptional regulatory sequence. Operably linked is intended to mean that the nucleotide sequence is linked to a regulatory sequence in a manner which allows expression of the nucleotide sequence. Regulatory sequences are art-recognized and are selected to direct expression of the subject proteins. Accordingly, the term transcriptional regulatory sequence includes promoters, enhancers and other expression control elements. Such regulatory sequences are described in Goeddel; Gene Expression Technology: Methods in Enzymology 185, Academic Press, San Diego, Calif. (1990). For instance, any of a wide variety of expression control sequences, sequences that control the expression of a DNA sequence when operatively linked to it, may be used in these vectors to express DNA sequences encoding the polypeptides of this invention. Such useful expression control sequences, include, for example, a viral LTR, such as the LTR of the Moloney murine leukemia virus, the early and late promoters of SV40, adenovirus or cytomegalovirus immediate early promoter, the lac system, the trp system, the TAC or TRC system, T7 promoter whose expression is directed by T7 RNA polymerase, the major operator and promoter regions of phage lambda., the control regions for fd coat protein, the promoter for 3-phosphoglycerate kinase or other glycolytic enzymes, the promoters of acid phosphatase, e.g., Pho5, the promoters of the yeast a-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other proteins encoded by the vector, such as antibiotic markers, should also be considered.

Moreover, the gene constructs of the present invention can also be used to deliver nucleic acids encoding an RNAi molecule of the present invention, or a target splicing variant, E2 or ΔE2. Thus, another aspect of the invention features expression vectors for in vivo or in vitro transfection and translation of AT1R splicing variants in particular cell types.

Expression constructs of a target splicing variant (E2 or ΔE2) may be administered in any biologically effective carrier, e.g. any formulation or composition capable of effectively delivering the recombinant gene to cells in vivo or in vitro. Approaches include insertion of the subject gene in viral vectors including recombinant retroviruses, adenovirus, adeno-associated virus, and herpes simplex virus-1, or recombinant bacterial or eukaryotic plasmids. Viral vectors transfect cells directly; plasmid DNA can be delivered with the help of, for example, cationic liposomes (lipofectin) or derivatized (e.g., antibody-conjugated), polylysine conjugates, gramacidin S, artificial viral envelopes or other such intracellular carriers, as well as direct injection of the gene construct or CaPO₄ precipitation. One of skill in the art can readily select from amongst available vectors and methods of delivery in order to optimize transfection into a particular cell type or under particular conditions.

A preferred approach for introduction of nucleic acid into a cell is by use of a viral vector containing nucleic acid, e.g., a CDNA, encoding an RNAi molecule of the present invention, or a target splicing variant (E2 or ΔE2). Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up the viral vector.

Retrovirus vectors and adeno-associated virus vectors are generally understood to be the recombinant gene delivery system of choice for the transfer of exogenous genes. These vectors provide efficient delivery of genes into cells, and the transferred nucleic acids are stably integrated into the chromosomal DNA of the host. A major prerequisite for the use of retroviruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A. D. (1990) Blood 76: 271). Thus, recombinant retrovirus can be constructed in which part of the retroviral coding sequence (gag, pol, env) has been replaced by nucleic acid encoding one of the subject proteins rendering the retrovirus replication-defective. The replication-defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines for preparing both ecotropic and amphotropic retroviral systems include ψCrip, ψCre, ψ2 and ψAm. Retroviruses have been used to introduce a variety of genes into many different cell types, including neuronal cells, in vitro and/or in vivo (see for example Eglitis, et al. (1985) Science 230: 1395-1398; Danos and Mulligan (1988) Proc. Natl. Acad. Sci. USA 85: 6460-6464; Wilson et al. (1988) Proc. Natl. Acad. Sci. USA 85: 3014-3018; Armentano et al. (1990) Proc. Natl. Acad. Sci. USA 87: 6141-6145; Huber et al. (1991) Proc. Natl. Acad. Sci. USA 88: 8039-8043; Ferry et al. (1991) Proc. Natl. Acad. Sci. USA 88: 8377-8381; Chowdhury et al. (1991) Science 254: 1802-1805; van Beusechem et al. (1992) Proc. Natl. Acad. Sci. USA 89: 7640-7644; Kay et al. (1992) Human Gene Therapy 3: 641-647; Dai et al. (1992) Proc. Natl. Acad. Sci. USA 89: 10892-10895; Hwu et al. (1993) J. Immunol. 150: 4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Furthermore, it has been shown that it is possible to limit the infection spectrum of retroviruses and consequently of retroviral-based vectors, by modifying the viral packaging proteins on the surface of the viral particle (see, for example PCT publications WO93/25234 and WO94/06920). For instance, strategies for the modification of the infection spectrum of retroviral vectors include: coupling antibodies specific for cell surface antigens to the viral env protein (Roux et al. (1989) PNAS 86: 9079-9083; Julan et al. (1992) J. Gen Virol 73: 3251-3255; and Goud et al. (1983) Virology 163: 251-254); or coupling cell surface receptor ligands to the viral env proteins (Neda et al. (1991) J Biol Chem 266: 14143-14146). Coupling can be in the form of the chemical cross-linking with a protein or other variety (e.g., lactose to convert the env protein to an asialoglycoprotein), as well as by generating fusion proteins (e.g., single-chain antibody/env fusion proteins). This technique, while useful to limit or otherwise direct. the infection to certain tissue types, can also be used to convert an ecotropic vector in to an amphotropic vector.

Moreover, use of retroviral gene delivery can be further enhanced by the use of tissue- or cell-specific transcriptional regulatory sequences which control expression of the gene of the retroviral vector.

Another viral gene delivery system useful in the present invention utilizes adenovirus-derived vectors. The genome of an adenovirus can be manipulated such that it encodes and expresses a gene product of interest but is inactivated in terms of its ability to replicate in a normal lytic viral life cycle. See for example Berkner et al. (1988) BioTechniques 6: 616; Rosenfeld et al. (1991) Science 252: 431-434; and Rosenfeld et al. (1992) Cell 68: 143-155. Suitable adenoviral vectors derived from the adenovirus strain Ad type 5 dl324 or other strains of adenovirus (e.g., Ad2, Ad3, Ad7 etc.) are well known to those skilled in the art. Recombinant adenoviruses can be advantageous in certain circumstances in that they can be used to infect a wide variety of cell types, including airway epithelium (Rosenfeld et al.(1992) cited supra), endothelial cells (Lemarchand et al. (1992) Proc. Natl. Acad. Sci. USA 89: 6482-6486), hepatocytes (Herz and Gerard (1993) Proc. Natl. Acad. Sci. USA 90: 2812-2816) and muscle cells (Quantin et al. (1992) Proc. Natl. Acad. Sci. USA 89: 2581-2584). Furthermore, the virus particle is relatively stable and amenable to purification and concentration, and as above, can be modified so as to affect the spectrum of infectivity.

Yet another viral vector system useful for delivery a desired RNAi molecule or nucleic acid encoding a target splicing variant (E2 or ΔE2) in the present invention is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al. Curr. Topics in Micro. and Immunol. (1992) 158: 97-129). It is also one of the few viruses that may integrate its DNA into non-dividing cells, and exhibits a high frequency of stable integration (see for example Flotte et al. (1992) Am. J. Respir. Cell. Mol. Biol. 7: 349-356; Samulski et al. (1989) J. Virol. 63: 3822-3828; and McLaughlin et al. (1989) J.Virol. 62: 1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5: 3251-3260 can be used to introduce DNA into cells. A variety of nucleic acids have been introduced into different cell types using AAV vectors (see for example Hermonat et al. (1984) Proc. Natl. Acad. Sci. USA 81: 6466-6470; Tratschin et al. (1985) Mol. Cell. Biol. 4: 2072-2081; Wondisford et al. (1988) Mol. Endocrinol. 2: 32-39; Tratschin et al. (1984) J. Virol. 51: 611-619; and Flotte et al. (1993) J. Biol. Chem. 268: 3781-3790).

The above cited examples of viral vectors are by no means exhaustive. Herpes-simplex viral vectors and lentiviral vectors are just two additional types of viral vectors which can be used in the present invention.

In addition to viral transfer methods, such as those illustrated above, non-viral methods can also be employed to cause expression of an exogenous nucleic acid of the present invention in cells or animals. Most nonviral methods of gene transfer rely on normal mechanisms used by cells for the uptake and intracellular transport of macromolecules. In preferred embodiments, non-viral gene delivery systems of the present invention rely on endocytic pathways for the uptake of an exogenous nucleic acid, e.g., an RNAi molecule of the present invention, by the targeted cell. Exemplary gene delivery systems of this type include liposomal derived systems, poly-lysine conjugates, and artificial viral envelopes.

III. Pharmaceutical Compositions and Uses Thereof

In another aspect, the present invention provides pharmaceutical preparations comprising, as an active ingredient, an agent capable of regulating AT1R posttranscriptionally such as described herein, formulated in an amount sufficient to alleviate, in vivo, high blood pressure or other biological consequences of aberrant angiotensin-mediated signaling.

The present invention also pertains to pharmaceutical compositions comprising the therapeutic agents identified by methods described herein. A therapeutic agent of the present invention can be formulated with a physiologically acceptable medium to prepare a pharmaceutical composition. The particular physiological medium may include, but is not limited to, water, buffered saline, polyols (e.g., glycerol, propylene glycol, liquid polyethylene glycol) and dextrose solutions. The optimum concentration of the active ingredient(s) in the chosen medium can be determined empirically, according to well known procedures, and will depend on the ultimate pharmaceutical formulation desired. Preferably, the composition is non-pyrogenic, i.e., does not substantially elevate the body temperature of a patient to whom it is administered.

One aspect of the invention is drawn to RNAi constructs and siRNAs, pharmaceutically acceptable salts of such constructs, and other bioequivalents. Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,NI-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. of Pharma Sci., 1977, 66,1-19). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the preparations of the invention. These include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids.

For siRNA oligonucleotides, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalene disulfonic acid, polygalacturonic acid, and the like; and (d) salts formed from elemental anions such as chlorine, bromine, and iodine.

Methods of introducing therapeutic agents at the site of treatment include, but are not limited to, intradermal, intramuscular, intraperitoneal, intravenous, subcutaneous, oral and intranasal. Other suitable methods of introduction can also include rechargeable or biodegradable devices and slow release polymeric devices. The pharmaceutical compositions of this invention can also be administered as part of a combinatorial therapy with other agents, or with other treatment methods.

The agents for use in the subject method may be conveniently formulated for administration with a biologically acceptable medium, such as water, buffered saline, polyol (for example, glycerol, propylene glycol, liquid polyethylene glycol and the like) or suitable mixtures thereof. The optimum concentration of the active ingredient(s) in the chosen medium can be determined empirically, according to procedures well known to medicinal chemists. As used herein, “biologically acceptable medium” includes any and all solvents, dispersion media, and the like which may be appropriate for the desired route of administration of the pharmaceutical preparation. The use of such media for pharmaceutically active substances is known in the art. Except insofar as any conventional media or agent is incompatible with the activity of the subject inhibitor agents, its use in the pharmaceutical preparation of the invention is contemplated. Suitable vehicles and their formulation inclusive of other proteins are described, for example, in the book Remington's Pharmaceutical Sciences (“Remington's Pharmaceutical Sciences,” Mack Publishing Company, Easton, Pa., U.S.A., 1985). These vehicles include injectable “deposit formulations.“Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of an active ingredient at a particular target site.

The preparations of the present invention may be given orally, parenterally, topically, or rectally. They are of course given by forms suitable for each administration route. For example, they are administered in tablets or capsule form, by injection, inhalation, eye lotion, ointment, suppository, controlled release patch, etc., administration by injection, infuision or inhalation; topical by lotion or ointment; and rectal by suppositories.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrastemal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

Regardless of the route of administration selected, the compounds of the present invention, which may be used in a suitable hydrated form, and/or the pharmaceutical compositions of the present invention, are formulated into pharmaceutically acceptable dosage forms such as described below or by other conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular inhibitor agent employed, the age, sex, weight, condition, general health and prior medical history of the patient being treated, and like factors well known in the medical arts.

The term “treatment” is intended to encompass also prophylaxis, therapy, and cure.

Treatment of these animals with test compounds will involve administration of the compound, in an appropriate form, to the animal. Administration will be by a route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated are systemic intravenous injection and regional administration via blood or lymph supply.

The patient receiving this treatment is any animal in need, including primates, in particular humans.

Accordingly, the present invention relates to a new method of preventing and/or treating cardiovascular diseases and complications by pharmacological interference with the RAS using an agent that regulate AT1R post-transcriptionally.

In one embodiment, the present invention relates to use of an agent capable of post-transcriptional regulation of AT1R in the manufacture of a medicament for the prophylactic and/or therapeutic treatment of angiotensin II-related diseases. Such diseases may include, without limitation, hypertension, cardiac hypertrophy, myocardial infarction, normal tension glaucoma, disorders on account of neurological pathogenesis, other cardiovascular complications including stroke, vascular access dysfimction and amputations, and cardiovascular complications encountered during or between dialysis of a patient in need of such dialysis.

A further embodiment of the invention provides a method for prophylactic and/or therapeutic treatment of a patient having or being at high risk for an angiotensin II-related disease, comprising administering to the patient having such a disease or being at such a risk a therapeutically effective amount of the medicament or pharmaceutical preparation of the present invention. Such diseases may include, without limitation, hypertension, cardiac hypertrophy, myocardial infarction, normal tension glaucoma, disorders on account of neurological pathogenesis, other cardiovascular complications including stroke, vascular access dysfunction and amputations, and cardiovascular complications encountered during or between dialysis of a patient in need of such dialysis.

### EXAMPLES

### Example 1

### In Vitro Translation

ΔE2 (ΔE2) and E2 (E2) cDNA were subcloned into the pcDNA5/FRT expression vector (Invitrogen). ΔE2 and E2 plasmid DNA was linearized by Xho I digestion. After confirming the digestion was complete on an agarose gel, linearized DNA was purified by the PCR purification system (Qiagen), then in vitro transcribed into capped RNA (T7 mMessage mMachine, Ambion). Transcribed RNAs were quantified using the Ribogreen RNA quantitation kit (Molecular Probes). RNA (0.5-2 μg) was translated in wheat germ extracts in the presence of [35S] Methionine (Promega). The translated protein was analyzed by SDS-gel electrophoresis. Protein size was determined by comparison with broad-range prestained SDS-PAGE markers. Autoradiograms of SDS gels were quantified by phosphorimaging and expressed in arbitrary units.

### Example 2

### A10 Cell Culture and Transient Transfections

A10 cells were cultured in Dulbecco's Modified Essential Medium (DMEM) supplemented with 2 mM L-glutamine, 10% fetal bovine serum and antibiotics (100 IU/ml penicillin, 100 μg/ml streptomycin) (DMEM media). When cells were 60-75% confluent, 20 μg of ΔE2- or E2-plasmid DNA per 100 mm dish was transiently transfected by the calcium phosphate method (Calcium phosphate transfection system, Invitrogen).

### Example 3

### AT1R Radioligand-Binding

After washing and pelleted the cells in phosphate buffered saline at 4° C., A10 cell membranes were prepared as described previously. After being re-suspended in Homogenization buffer [10 mM Tris buffer (pH 7.2) containing 0.32 M sucrose, 2 mM EDTA, and 3 mM MgCl₂], the cells were briefly homogenized with a polytron (PT-10, Brinkmann Instruments). The homogenate was centrifuged at 1000×g for 5 min and the resulting supernatant, at 44,000×g for 65 min. The final pellet was re-suspended in Binding buffer [10 mM Tris (pH 7.2) containing 3 mM MgCl₂] and the protein content determined using the Bio-Rad DC protein assay. Membrane protein was adjusted to the appropriate concentration in Binding buffer supplemented with 0.2% bovine serum albumin and stored at −80° C. until use in the radioligand binding assay. Glomeruli were isolated as described previouslyl8. Both A10 membranes (15 μg protein/tube) and isolated glomeruli (5 μg protein/tube) were incubated for 1-2 h at room temperature with increasing concentrations of ¹²⁵1-[Sarl,Ile8] angiotensin II (Peptide Radioiodination Ctr, Pullman, Wash.) in the presence of 1 μM PD-123319, an AT2R antagonist (so only rAT1R expression was measured). Binding reactions were terminated by rapid filtration through a Brandel cell harvester. Quantitation: Bound radioligand was measured in a 7-spectrophotometer. Specific ATI receptor binding was defined as the total amount of radioligand bound minus the nonspecific binding, defined as the amount bound in the presence of 200 nM Angiotensin II (100×Kd for angiotensin II). Data points were obtained in triplicate. Kd and Bmax values from Scatchard plots were determined using the nonlinear regression analysis program, PRISM.

### Example 4

### Inositol Phosphate Assay

A10 cells were cultured to 65% confluence in 24-well plates in DMEM media before being transiently transfected with rATla E2 or ΔE2 plasmid DNA (using the same ratio of DNA/cells for each transcript). After 2 days, the cells were treated for 16 hours with DMEM media containing 3 μCi/ml myo-[³H]-inositol (Amersham). After washing twice, the cells were preincubated with 500 μl of DMEM media supplemented with 10 mM LiCl for 15 min before stimulation with Angiotensin II (10⁻¹⁰ to 10⁻⁶ M) for 20 min at 37° C. The incubation was terminated by adding 750 μl/well of ice-cold 10 mM formic acid, followed by a 30 min incubation on ice. [³H]Inositol phosphates were eluted from ion exchange columns (AG-1-X8 resin, 200-400 mesh, BioRad) using 0.1 M formic acid/0.8 M ammonium formate. Radioactive IP fractions were counted in a Beckman scintillation counter.

### Example 5

### Animal Models

Adult female DS and DR rats (175-200 g) were maintained on a phytoestrogen-free, high sodium diet (NaCl=7.6%; Harlan) with free access to water for 4 weeks before mean arterial pressure (MAP), glomerular rAT1R density and expression levels of rAT1aR splice variants were determined.

Mean Arterial Pressure: Animals were anesthetized with Inactin (100 mg/kg, ip) and catheters were placed in the carotid artery for MAP measurements essentially.

### Example 6

### Real-Time PCR

Total RNA was extracted using TRIzol reagent (Life Technologies). First strand cDNA was made from total RNA using the AMV reverse transcriptase system (Promega) with random hexamers. Quantitations of specific mRNAs and 18S rRNA (for control) were performed by real-time PCR using the ABI Prism 7700 Sequence Detection System (Perkin Elmer Applied Biosystems). The PCR reaction mixture consisted of RNase free water, TaqMan Universal PCR Master Mix (Perkin Elmer Applied Biosystems) and 300 nM specific primers and 10 μM probe (Forward primers: 119F (E2), 5′-CCA CAT TCC CTG AGT TAA CAT ATG A-3′ and 114F (ΔE2), 5′-CTC TGC CAC ATT CCC TGG TC-3′; Reverse primer: 310R (E2 & ΔE2), 5′-TCT TTT GAT ACC ATC TTC AGC AGA A-3′); and Probe: 232T (E2 & ΔE2), 6 FAM-TCG AAT AGT GTC TGA GAC CAA CTC AAC CCA-TAMRA), and cDNA samples. PCR conditions were optimized for the probe (232T) and both sets of primers (119F & 310R and 114F & 310R) using control cDNAs. The expression of mRNA and 18S rRNA in each sample was quantitated using respective primers. PCR reactions without reverse transcription were included to control for contamination by genomic DNA. The standard curves for 18S rRNA and mRNA were made by a series of ten times dilutions (5³, 5⁴, 5⁵, 5⁶, 5⁷, and 58) of the cDNA. The standard curves were calculated based on the control values.

### Example 7

### ΔE2 RNA is Translated into rAT1R Protein More Efficiently than E2

ΔE2 and E2 plasmid DNA (pcDNA5/FRT) was linearized by Xho I digestion then in vitro transcribed into capped RNA. RNA (0.5-2 μg) was translated in wheat germ extracts in the presence of [³⁵S] methionine and analyzed by phosphorimaging of autoradiograms of SDS gels. These in vitro studies revealed that 1.8-fold more rAT1R protein (40 KDa) was synthesized by the ΔE2 transcript compared to the E2 variant.

### Example 8

### The Density of rAt1aRs is Markedly Higher in A10 Cells Transfected with ΔE2 Compared to E2 Plasmid DNA

The A10 rat aortic smooth muscle cell line was transiently transfected by calcium phosphate with ΔE2 or E2 plasmid DNA for 2 days before membranes were isolated for radioligand binding analysis. These transfection experiments revealed that the rAT1R density (Bmax) was 40% higher in A1O cells transfected with the ΔE2 plasmid compared to the E2.

### Example 9

### rAt1aR Signaling is Markedly Higher in A10 Cells Transfected with ΔE2 Compared to E2 Plasmid DNA

A10 cells were transiently transfected with ΔE2 or E2 plasmid cDNA for two days before loading cells with [³H]-inositol. Cells transfected with the ΔE2 plasmid accumulated 33% more inositol phosphates than E2 transfected cells after stimulation with 100 nM angiotensin II for 20 min (angiotensin IIbasal: ΔE2, 1.8+0.2; E2, 1.35+0.2, n=4, p<0.05).

### Example 10

### AT1R Densities are Elevated in the Hypertensive DS Rat Kidney

Female DS and DR rats were maintained on a normal salt (NS, 0.4% NaCl) and high salt (HS, 7.6% NaCl) diet for 4 weeks. The HS diet significantly increased the MAP in the DS rats compared to the NS diet but had no significant effects on MAP in the DR rats. No significant differences in MAP were found between the DS and DR animals maintained on the NS diet. On the HS diet, glomerular AT1R density was significantly increased (by 50%) in the DS rats maintained on the HS diet compared to the DR animals and by 15% compared to the DS animals maintained on the NS diet. No significant differences in AT1R expression were found between the DR rats maintained on the NS and HS diets. Furthermore, on the NS diet, the DS rats had a significantly higher level of glomerular AT1R density compared to the DR animal maintained on either the HS or NS diets. These findings were similar to AT1R binding to membranes prepared from renal cortex, although AT1R densities were 10-fold less in whole renal cortex preparations compared to isolated glomeruli preparations.

### Example 11

### The Hypertensive DS Rat Expresses a Higher Percentage of Renal ΔE2/Total AT1aR mRNA Compared to Normotensive DR and DS Rats

Total RNA was isolated from the renal cortex of DR and DS animals maintained on NS and HS diets and reverse transcribed before real-time PCR was performed using primers specific to the ΔE2 and E2 splice variants. Two rAt1aR alternative splice transcripts (ΔE2 and E2) were amplified. Although no significant differences were observed in the total levels of At1aR mRNA among all four animal groups (data not shown), the ratio of ΔE2/E2 was 57% higher in the hypertensive DS rat compared to the normotensive DR animal maintained on the HS diet. This increase in the ratio of the splice variant lacking E2 was not due to increased ΔE2 expression but rather a decrease in the E2 transcript.

The publications and other materials used herein to illuminate the background of the invention, and in particular, cases to provide additional details respecting the practice, are incorporated by reference in full.

### Equivalents

Those skilled in the art will recognize, or be able to ascertain using no more than routine experimentation, many equivalents to the specific embodiments of the invention described herein. Such equivalents are intended to be encompassed by the following claims.

1. A method of screening for agents that reduce angiotensin II-mediated signaling in mammals, comprising conducting an in vitro or cell-based assay with a plurality of candidate agents and 1) identifying agents that increase the ratio of AT1R splicing variant with exon 2 versus AT1R splicing variant without exon 2; or 2) identifying agents that promote the translation of AT1R splicing variant with exon 2, or inhibit the translation of AT1R splicing variant without exon 2, or both 1) and 2).

2. The method of claim 1, wherein said agents are siRNAs.

3. The method of claim 3, wherein said siRNA targets the RNA juncture between exons 1 and 3 of AT1R splicing variant without exon 2.

4. The method of claim 1, wherein said agents are small molecules.

5. The method of claim 1, wherein said agents modulate the activities of RNA binding proteins that interact with 5′ leader sequence of AT1R mRNA.

6. The method of claim 1, wherein said assay is an in vitro translation assay using AT1R splicing variants with or without exon 2 as templates to be translated.

7. The method of claim 1, wherein said assay is an in vitro splicing assay using AT1R gene as a template or part of the AT1R gene in combination with a reporter gene.

8. The method of claim 1, wherein said assay is a cell-based assay measuring AT1R binding by angiotensin II.

9. The method of claim 1, wherein said assay is a cell-based assay measuring angiotensin II-mediated signaling.

10. The method of claim 9, wherein said signaling results in the production of inositol phosphate.

11. A pharmaceutical preparation comprising an agent that inhibits the alternative splicing resulting in AT1R transcripts without exon 2, promotes the alternative splicing resulting in AT1R transcript with exon 2, or both.

12. A pharmaceutical preparation comprising an agent that promotes the translation of AT1R splicing variant with exon 2, or inhibits the translation of AT1R splicing variant without exon 2, or both.

13. A method of using the pharmaceutical preparation of claim 11 or 12 to treat an angiotensin II-mediated disease in a mammal, comprising administering said pharmaceutical preparation to patients having said disease.

14. An expression vector comprising a nucleic acid encoding an siRNA or a hairpin RNA that specifically targets the RNA juncture between exons 1 and 3 of AT1R.

15. An expression vector comprising a nucleic acid encoding an antisense sequence that attenuates the expression of AT1R splicing variant without exon 2.


SEQUENCE LISTING (64 sequences)

SEQ ID NO: 1 - DNA, 2347 bp, Homo sapiens
attcggagctgcctcctcgccaatgattccagcgcctgacagccaggaccccaggcagcagcgagtgacaggacgtctggaccggcgcgccgctagcagctctgccgggccgcggcggtgatcgatggggagcggctggagcggacccagcgagtgagggcgcacagccgggacgccgaggcggcgggcgggagacccgcaccagcgcagccggccctcggcgggacgtgacgcagcgcccggggcgcgggtttgatatttgacaaattgatctaaaatggctgggtttttatctgaataactcactgatgccatcccagaaagtcggcaccaggtgtatttgatatagtgtttgcaacaaattcgacccaggtgatcaaaatgattctcaactcttctactgaagatggtattaaaagaatccaagatgattgtcccaaagctggaaggcataattacatatttgtcatgattcctactttatacagtatcatctttgtggtgggaatatttggaaacagcttggtggtgatagtcatttacttttatatgaagctgaagactgtggccagtgtttttcttttgaatttagcactggctgacttatgctttttactgactttgccactatgggctgtctacacagctatggaataccgctggccctttggcaattacctatgtaagattgcttcagccagcgtcagtttcaacctgtacgctagtgtgtttctactcacgtgtctcagcattgatcgatacctggctattgttcacccaatgaagtcccgccttcgacgcacaatgcttgtagccaaagtcacctgcatcatcatttggctgctggcaggcttggccagtttgccagctataatccatcgaaatgtatttttcattgagaacaccaatattacagtttgtgctttccattatgagtcccaaaattcaacccttccgatagggctgggcctgaccaaaaatatactgggtttcctgtttccttttctgatcattcttacaagttatactcttatttggaaggccctaaagaaggcttatgaaattcagaagaacaaaccaagaaatgatgatatttttaagataattatggcaattgtgcttttctttttcttttcctggattccccaccaaatattcacttttctggatgtattgattcaactaggcatcatacgtgactgtagaattgcagatattgtggacacggccatgcctatcaccatttgtatagcttattttaacaattgcctgaatcctcttttttatggctttctggggaaaaaatttaaaagatattttctccagcttctaaaatatattcccccaaaagccaaatcccactcaaacctttcaacaaaaatgagcacgctttcctaccgcccctcagataatgtaagctcatccaccaagaagcctgcaccatgttttgaggttgagtgacatgttcgaaacctgtccataaagtaattttgtgaaagaaggagcaagagaacattcctctgcagcacttcactaccaaatgagcattagctacttttcagaattgaaggagaaaatgcattatgtggactgaaccgacttttctaaagctctgaacaaaagcttttctttccttttgcaacaagacaaagcaaagccacattttgcattagacagatgacggctgctcgaagaacaatgtcagaaactcgatgaatgtgttgatttgagaaattttactgacagaaatgcaatctccctagcctgcttttgtcctgttattttttatttccacataaaggtatttagaatatattaaatcgttagaggagcaacaggagatgagagttccagattgttctgtccagtttccaaagggcagtaaagttttcgtgccggttttcagctattagcaactgtgctacacttgcacctggtactgcacattttgtacaaagatatgctaagcagtagtcgtcaagttgcagatctttttgtgaaattcaacctgtgtcttataggtttacactgccaaaacaatgcccgtaagatggcttatttgtataatggtgttactaaagtcacatataaaagttaaactacttgtaaaggtgctgcactggtcccaagtagtagtgtcctcctagtatattagtttgatttaatatctgagaagtgtatatagtttgtggtaaaaagattatatatcataaagtatgccttcctgtttaaaaaaagtatatattctacacatatatatatatgtatatctatatctctaaactgctgttaattgattaaaatctggcaaagttatatttactttaaaataaaataattttattgc

SEQ ID NO: 2 - DNA, 21 bp, Artificial Sequence
  Target No. 1 5′ to 3′
tctgccacattccctggtcaa

SEQ ID NO: 3 - DNA, 21 bp, Artificial Sequence
  Sense strand of siRNA to Target No. 1
tgccacauugggaggugaauu

SEQ ID NO: 4 - RNA, 21 nt, Artificial Sequence
  Anti-sense strand of siRNA to Target No. 1
uugaccugggaauguggcaga

SEQ ID NO: 5 - DNA, 21 bp, Artificial Sequence
  Target No. 2 5′ to 3′
ctgccacattccctggtcaag

SEQ ID NO: 6 - RNA, 21 nt, Artificial Sequence
  Sense strand of siRNA to Target No. 2
gccacauucccuggucaaguu

SEQ ID NO: 7 - RNA, 21 nt, Artificial Sequence
  Anti-sense strand of siRNA to Target No. 2
cuugaccugggaauguggcag

SEQ ID NO: 8 - DNA, 21 bp, Artificial Sequence
  Target No. 3  5′ to 3′
tgccacattccctggtcaagt

SEQ ID NO: 9 - RNA, 21 nt, Artificial Sequence
  Sense strand of siRNA to Target No. 3
ccacauucccuggucaaguuu

SEQ ID NO: 10 - RNA, 21 nt, Artificial Sequence
  Anti-sense strand of siRNA to Target No. 3
acuugaccugggaauguggca

SEQ ID NO: 11 - DNA, 21 bp, Artificial Sequence
  Target No. 4 5′ to 3′
gccacattccctggtcaagtg

SEQ ID NO: 12 - RNA, 21 nt, Artificial Sequence
  Sense strand of siRNA to Target No. 4
cacauucccuggucaaguguu

SEQ ID NO: 13 - RNA, 21 nt, Artificial Sequence
  Anti-sense strand of siRNA to Target No. 4
gacuugaccugggaauguggc

SEQ ID NO: 14 - DNA, 21 bp, Artificial Sequence
  Target No. 5 5′ to 3′
ccacattccctggtcaagtgg

SEQ ID NO: 15 - RNA, 21 nt, Artificial Sequence
  Sense strand of siRNA to Target No. 5
acauucccuggucaagugguu

SEQ ID NO: 16 - RNA, 21 nt, Artificial Sequence
  Anti-sense strand of siRNA to Target No. 5
ccacuugaccugggaaugugg

SEQ ID NO: 17 - DNA, 21 bp, Artificial Sequence
  Target No. 6 5′ to 3′
cacattccctggtcaagtgga

SEQ ID NO: 18 - RNA, 21 nt, Artificial Sequence
  Sense strand of siRNA to Target No. 6
cauucccuggucaaguggauu

SEQ ID NO: 19 - DNA, 21 bp, Artificial Sequence
  Anti-sense strand of siRNA to Target No. 6
tccacuugaccugggaaugug

SEQ ID NO: 20 - DNA, 21 bp, Artificial Sequence
  Target No. 7 5′ to 3′
acattccctggtcaagtggat

SEQ ID NO: 21 - RNA, 21 nt, Artificial Sequence
  Sense strand of siRNA to Target No. 7
auucccuggucaaguggauuu

SEQ ID NO: 22 - DNA, 21 bp, Artificial Sequence
  Anti-sense strand of siRNA to Target No. 7
atccacuugaccugggaaugu

SEQ ID NO: 23 - DNA, 21 bp, Artificial Sequence
  Target No. 8 5′ to 3′
cattccctggtcaagtggatt

SEQ ID NO: 24 - RNA, 21 nt, Artificial Sequence
  Sense strand of siRNA to Target No. 8
uucccuggucaaguggauuuu

SEQ ID NO: 25 - DNA, 21 bp, Artificial Sequence
  Anti-sense strand of siRNA to Target No. 8
aatccacuugaccugggaaug

SEQ ID NO: 26 - DNA, 21 bp, Artificial Sequence
  Target No. 9 5′ to 3′
attccctggtcaagtggattt

SEQ ID NO: 27 - RNA, 21 nt, Artificial Sequence
  Sense strand of siRNA to Target No. 9
ucccuggucaaguggauuuuu

SEQ ID NO: 28 - DNA, 21 bp, Artificial Sequence
  Anti-sense strand of siRNA to Target No. 9
aaatccacuugaccugggaau

SEQ ID NO: 29 - DNA, 21 bp, Artificial Sequence
  Target No. 10 5′ to 3′
ttccctggtcaagtggatttc

SEQ ID NO: 30 - RNA, 21 nt, Artificial Sequence
  Sense strand of siRNA to Target No. 10
cccuggucaaguggauuucuu

SEQ ID NO: 31 - DNA, 21 bp, Artificial Sequence
  Anti-sense strand of siRNA to Target No. 10
gaaatccacuugaccugggaa

SEQ ID NO: 32 - DNA, 21 bp, Artificial Sequence
  Target No. 11 5′ to 3′
tccctggtcaagtggatttcg

SEQ ID NO: 33 - RNA, 21 nt, Artificial Sequence
  Sense strand of siRNA to Target No. 11
ccuggucaaguggauuucguu

SEQ ID NO: 34 - DNA, 21 bp, Artificial Sequence
  Anti-sense strand of siRNA to Target No. 11
cgaaatccacuugaccuggga

SEQ ID NO: 35 - DNA, 21 bp, Artificial Sequence
  Target No. 12 5′ to 3′
ccctggtcaagtggatttcga

SEQ ID NO: 36 - RNA, 21 nt, Artificial Sequence
  Sense strand of siRNA to Target No. 12
cuggucaaguggauuucgauu

SEQ ID NO: 37 - DNA, 21 bp, Artificial Sequence
  Anti-sense strand of siRNA to Target No. 12
ucgaaatccacuugaccuggg

SEQ ID NO: 38 - DNA, 21 bp, Artificial Sequence
  Target No. 13 5′ to 3′
cctggtcaagtggatttcgaa

SEQ ID NO: 39 - RNA, 21 nt, Artificial Sequence
  Sense strand of siRNA to Target No. 13
uggucaaguggauuucgaauu

SEQ ID NO: 40 - DNA, 21 bp, Artificial Sequence
  Anti-sense strand of siRNA to Target No. 13
uucgaaatccacuugaccugg

SEQ ID NO: 41 - DNA, 22 bp, Artificial Sequence
  Target No. 14 5′ to 3′
acattccctggtcaagtggatt

SEQ ID NO: 42 - RNA, 22 nt, Artificial Sequence
  Sense strand of siRNA to Target No. 14
auucccuggucaaguggauuuu

SEQ ID NO: 43 - DNA, 22 bp, Artificial Sequence
  Anti-sense strand of siRNA to Target No. 14
aatccacuugaccugggaaugu

SEQ ID NO: 44 - DNA, 22 bp, Artificial Sequence
  Target No. 15 5′ to 3′
cattccctggtcaagtggattt

SEQ ID NO: 45 - RNA, 22 nt, Artificial Sequence
  Sense strand of siRNA to Target No. 15
uucccuggucaaguggauuuuu

SEQ ID NO: 46 - DNA, 22 bp, Artificial Sequence
  Anti-sense strand of siRNA to Target No. 15
aaatccacuugaccugggaaug

SEQ ID NO: 47 - DNA, 23 bp, Artificial Sequence
  Target No. 16 5′ to 3′
cacattccctggtcaagtggatt

SEQ ID NO: 48 - RNA, 23 nt, Artificial Sequence
  Sense strand of siRNA to Target No. 16
gauucccuggucaaguggauuuu

SEQ ID NO: 49 - DNA, 23 bp, Artificial Sequence
  Anti-sense strand of siRNA to Target No. 16
aatccacuugaccugggaaugug

SEQ ID NO: 50 - DNA, 23 bp, Artificial Sequence
  Target No. 17 5′ to 3′
cattccctggtcaagtggatttc

SEQ ID NO: 51 - RNA, 23 nt, Artificial Sequence
  Sense strand of siRNA to Target No. 17
uucccuggucaaguggauuucuu

SEQ ID NO: 52 - DNA, 23 bp, Artificial Sequence
  Anti-sense strand of siRNA to Target No. 17
gaaatccacuugaccugggaaug

SEQ ID NO: 53 - DNA, 24 bp, Artificial Sequence
  Target No. 18 5′ to 3′
ccacattccctggtcaagtggatt

SEQ ID NO: 54 - RNA, 24 nt, Artificial Sequence
  Sense strand of siRNA to Target No. 18
agauucccuggucaaguggauuuu

SEQ ID NO: 55 - DNA, 24 bp, Artificial Sequence
  Anti-sense strand of siRNA to Target No. 18
aatccacuugaccugggaaugugg

SEQ ID NO: 56 - DNA, 24 bp, Artificial Sequence
  Target No. 19 5′ to 3′
cattccctggtcaagtggatttcg

SEQ ID NO: 57 - RNA, 24 nt, Artificial Sequence
  Sense strand of siRNA to Target No. 19
uucccuggucaaguggauuucguu

SEQ ID NO: 58 - DNA, 24 bp, Artificial Sequence
  Anti-sense strand of siRNA to Target No. 19
cgaaatccacuugaccugggaaug

SEQ ID NO: 59 - DNA, 25 bp, Artificial Sequence
  Target No. 20 5′ to 3′
gccacattccctggtcaagtggatt

SEQ ID NO: 60 - RNA, 25 nt, Artificial Sequence
  Sense strand of siRNA to Target No. 20
cagauucccuggucaaguggauuuu

SEQ ID NO: 61 - DNA, 25 bp, Artificial Sequence
  Anti-sense strand of siRNA to Target No. 20
aatccacuugaccugggaauguggc

SEQ ID NO: 62 - DNA, 25 bp, Artificial Sequence
  Target No. 21 5′ to 3′
cattccctggtcaagtggatttcga

SEQ ID NO: 63 - RNA, 25 nt, Artificial Sequence
  Sense strand of siRNA to Target No. 21
uucccuggucaaguggauuucgauu

SEQ ID NO: 64 - DNA, 25 bp, Artificial Sequence
  Anti-sense strand of siRNA to Target No. 21
ucgaaatccacuugaccugggaaug